SEQUENCE LISTING (12 sequences)

SEQ ID NO: 1 - DNA, 20 bp, Unknown
  Synthetic primer sequence
ctgcccagtttcgaggtcat

SEQ ID NO: 2 - DNA, 20 bp, Unknown
  Synthetic primer sequence
caatcggaatgcgcttgagg

SEQ ID NO: 3 - DNA, 20 bp, Unknown
  Synthetic primer sequence
caggaaggtggctcttggag

SEQ ID NO: 4 - DNA, 20 bp, Unknown
  Synthetic primer sequence
cccatcctcagcatcgactc

SEQ ID NO: 5 - DNA, 21 bp, Unknown
  Synthetic primer sequence
tgtagcaactggcttagttca

SEQ ID NO: 6 - DNA, 21 bp, Unknown
  Synthetic primer sequence
tggccacagaggagtctctta

SEQ ID NO: 7 - DNA, 21 bp, Unknown
  Synthetic primer sequence
cgcgagaagatgacccagatc

SEQ ID NO: 8 - DNA, 20 bp, Unknown
  Synthetic primer sequence
gaggcgtacagggatagcac

SEQ ID NO: 9 - DNA, 20 bp, Unknown
  Synthetic primer sequence
aagcatcaacacacccaaca

SEQ ID NO: 10 - DNA, 20 bp, Unknown
  Synthetic primer sequence
cttgagctccattcgtgaca

SEQ ID NO: 11 - DNA, 20 bp, Unknown
  Synthetic primer sequence
ataggcccatctgtctcccc

SEQ ID NO: 12 - DNA, 19 bp, Unknown
  Synthetic primer sequence
caggtggctgtctgaggaa

(19) United States
(12) Patent Application Publication
(10) Pub. No.: US 2022/0257633 A1
(43) Pub. Date: Aug. 18, 2022

(54) METHOD OF TREATING AGE-RELATED MACULAR DEGENERATION

(71) Applicant: THE PROVOST, FELLOWS, FOUNDATION SCHOLARS, & THE O, Dublin (IE)

(72) Inventors: SARAH DOYLE, DUBLIN (IE); MATTHEW CAMPBELL, DUBLIN (IE); EMA OZAKI, DUBLIN (IE); KELLY MULFAUL, DUBLIN (IE)

(21) Appl. No.: 17/671,688

(22) Filed: Feb. 15, 2022

Related U.S. Application Data
(60) Provisional application No. 63/150,177, filed on Feb. 17, 2021.

(51) Int. Cl. A61K 31/713; A61K 38/00; C12N 15/115; C12N 15/113; A61P 27/02

(52) U.S. Cl. CPC A61K 31/713; A61K 38/00; A61P 27/02; C12N 15/1138; C12N 15/115

(57) ABSTRACT

The present invention relates to a method of treating age-related macular degeneration. In particular embodiments, the invention relates to methods of treating age-related macular degeneration in a subject in need thereof, the method comprising the step of decreasing the expression or activation of a toll-like receptor in the subject.

### CROSS-REFERENCE TO RELATED APPLICATIONS

This application claims benefit of priority of U.S. Provisional Patent Application No. 63/150,177 entitled, “A METHOD OF TREATING AGE-RELATED MACULAR DEGENERATION”, filed Feb. 17, 2021. The entire contents and disclosures of these patent applications are incorporated herein by reference in their entirety.

### FIELD OF THE INVENTION

The present invention relates to a method of treating age-related macular degeneration. In particular embodiments, the invention relates to methods for treating dry age-related macular degeneration.

### BACKGROUND TO THE INVENTION

Age-related macular degeneration (AMD) is the leading cause of central blindness in adults. Dry AMD (also called nonexudative AMD) is a broad designation, encompassing forms of AMD that are not neovascular. At present, there are no treatments for dry AMD. Genetic factors, age, diet and smoking are risk factors for AMD.

The common, coding variant Y402H in the Complement Factor H (CFH) gene is strongly associated with influencing susceptibility to AMD. In fact, the role of complement in the retina is a topic of intense investigation, as it contributes to a variety of other retinal disease pathologies in addition to AMD. Variants in other complement-related genes are associated with AMD risk, including C3, CFI and C9, all result in an overly active complement system. Congruent with this, the deposition of C3, C5, and presence of membrane attack complex (MAC), have been demonstrated in donor eyes with early AMD. Accordingly, SNPs in complement factors account for ˜75% of genetic risk of developing AMD.

However, molecular triggers that initiate complement fixation in individuals with no apparent genetic risk remain unknown. Smoking is the largest modifiable risk factor for AMD, consequently oxidative stress has been implicated in disease. Genotype and smoking have been independently related to AMD with multiplicative joint effects, however, a tangible connection between the effects of oxidative stress and complement-associated pathology remains largely unidentified.

The retina is exposed to oxidative stress, which refers to cellular damage caused by reactive oxygen species (ROS), due to its high consumption of oxygen, its high proportion of polyunsaturated fatty acids, and its exposure to visible light. Excessive oxidative stress induces deleterious changes that result in visual impairment. AMD is a leading causes of visual impairment and involvement of oxidative stress has been reported. Furthermore, oxidative stress is thought to contribute to loss of cone photoreceptors in rare inherited retinopathies after degeneration of rod photoreceptors. 2-(w-Carboxyethyl) pyrrole (CEP) is an oxidative-stress modification involved in promoting angiogenesis during wound healing. Excessive ROS can damage lipids through a mechanism known as lipid peroxidation and CEP modifications are generated by oxidation of docosahexaenoate (DHA)-containing lipids, which are found at high levels in the membrane of photoreceptor cells. Of note, CEP-adducted proteins and CEP-ethanolamine phospholipids (CEP-EPs) are found in abundance in eyes and serum of patients with AMD compared with age-matched controls.

Toll-like receptors (TLRs) are a family of membrane-bound pattern recognition receptors (PRRs) located either on the cell surface or in endosomal compartments. These receptors are known to respond to host-molecules termed damage-associated molecular patterns (DAMPs) that have taken on the appearance of “non-self”. Sterile inflammation occurs in response to a growing list of DAMPs ranging from oxidized lipids or lipoproteins, to deposits of protein/lipid aggregates or particulate matter. As these stimuli are often not easily cleared, they can persist causing over-activation of the immune system and contributing to disease pathogenesis. Ten human TLRs utilize four adaptor proteins to fine tune the response required; MyD88, Mal/TIRAP, TRAM and TRIF. Activation of TLRs leads to activation of a multitude of signaling pathways and transcription factors that determine the type and duration of the inflammatory response.

### SUMMARY OF THE INVENTION

According to a first aspect of the present invention there is provided a method of treating age-related macular degeneration in a subject in need thereof.

Optionally, the method comprises the step of decreasing the expression or activation of a toll-like receptor in the subject.

Optionally, the toll-like receptor is selected from TLR1, TLR2, TLR3, TLR4, TLR5, TLR6, TLR7, TLR8, TLR9, TLR10, TLR11, TLR12, and TLR13.

Preferably, the toll-like receptor is TLR2.

Optionally, the method of treating age-related macular degeneration in a subject in need thereof comprises the step of decreasing the expression or activation of TLR2 in the subject.

Optionally, the age-related macular degeneration is dry age-related macular degeneration. Further optionally, the age-related macular degeneration is non-exudative age-related macular degeneration. Still further optionally, the age-related macular degeneration is non-neovascular age-related macular degeneration. Still further optionally, the age-related macular degeneration is not wet age-related macular degeneration.

Optionally, the method of treating dry age-related macular degeneration in a subject in need thereof comprises the step of decreasing the expression or activation of TLR2 in the subject.

Optionally, the method comprises the step of administering an antagonist of a toll-like receptor to the subject. Further optionally, the method comprises the step of administering a pharmaceutically effective amount of an antagonist of a toll-like receptor to the subject. Still further optionally, the method comprises the step of administering a pharmaceutically effective amount of an antagonist of a toll-like receptor to the subject to decrease the expression or activation of the toll-like receptor in the subject. Still further optionally, the method comprises the step of administering a pharmaceutically effective amount of an antagonist of a toll-like receptor to the subject to decrease the expression or activation of the toll-like receptor and so treat age-related macular degeneration in the subject.

Optionally, the method comprises the step of administering an antagonist of TLR2 to the subject. Further optionally, the method comprises the step of administering a pharmaceutically effective amount of an antagonist of TLR2 to the subject. Still further optionally, the method comprises the step of administering a pharmaceutically effective amount of an antagonist of TLR2 to the subject to decrease the expression or activation of TLR2 in the subject. Still further optionally, the method comprises the step of administering a pharmaceutically effective amount of an antagonist of TLR2 to the subject to decrease the expression or activation of TLR2 and so treat age-related macular degeneration in the subject.

Optionally, the method of treating dry age-related macular degeneration in a subject in need thereof comprises the step of administering a pharmaceutically effective amount of an antagonist of TLR2 to the subject to decrease the expression or activation of TLR2 and so treat age-related macular degeneration in the subject.

Optionally, the method comprises decreasing expression of the toll-like receptor. Further optionally, the method comprises decreasing expression of TLR2.

Optionally, the method comprises the step of administering an agent capable of decreasing expression of the toll-like receptor. Further optionally, the method comprises the step of administering an agent capable of decreasing expression of the toll-like receptor gene. Still further optionally, the method comprises the step of administering an agent capable of decreasing transcription of the toll-like receptor gene.

Optionally, the method comprises the step of administering an agent capable of decreasing expression of TLR2. Further optionally, the method comprises the step of administering an agent capable of decreasing expression of the TLR2 gene. Still further optionally, the method comprises the step of administering an agent capable of decreasing transcription of the TLR2 gene.

Optionally, the agent is selected from the group consisting of antisense oligonucleotides, ribozymes, small interfering RNAs (siRNA), microRNA (miRNA), small/small hairpin RNA (shRNA), and nucleic acid aptamers.

Optionally, the agent is a nucleic acid aptamer. Further optionally, the agent is a deoxyribonucleic acid aptamer. Further optionally, the agent is the deoxyribonucleic acid aptamer AP177 (as dislcosed in Y. C. Chang, W. C. Kao, W. Y. Wang, W. Y. Wang, R. B. Yang, K. Peck “Identification and characterization of oligonucleotides that inhibit Toll-like receptor 2-associated immune responses” FASEB J., 23 (2009), pp. 3078-3088).

Optionally, the agent is a vector. Further optionally, the agent is a viral vector. Still further optionally, the agent is a virally-delivered vector. Still further optionally, the agent is a retrovirus-, adenovirus-, herpes simplex-, vaccinia-, or adeno-associated virus-delivered vector.

Optionally, the agent is delivered by injection of naked DNA, electroporation, the gene gun, sonoporation, magnetofection, the use of oligonucleotides, lipoplexes, dendrimers, or inorganic nanoparticles.

Optionally, the method comprises decreasing the activation of the toll-like receptor. Further optionally, the method comprises decreasing the activation of TLR2.

Optionally, the method comprises administering a toll-like receptor antagonist selected from a competitive toll-like receptor antagonist, a non-competitive toll-like receptor antagonist, an uncompetitive toll-like receptor antagonist, a silent toll-like receptor antagonist, and an inverse toll-like receptor agonist.

Optionally, the method comprises administering a toll-like receptor antagonist selected from a reversible toll-like receptor antagonist, and an irreversible toll-like receptor antagonist.

Optionally, the method comprises administering a toll-like receptor antagonist selected from a selective toll-like receptor antagonist, and a non-selective toll-like receptor antagonist.

Optionally, toll-like receptor antagonist is selected from a chemical compound toll-like receptor antagonist, a small molecule toll-like receptor antagonist, an immunoglobulin toll-like receptor antagonist, and a lipid-A analogue toll-like receptor antagonist.

Optionally, the toll-like receptor antagonist is the small molecule toll-like receptor antagonist 2-ethoxy-1-({4-[2-(2H-1,2,3,4-tetrazol-5-yl)phenyl]phenyl}methyl)-1H-1,3-benzodiazole-7-carboxylic acid (candesartan cilexetil, “Atacand”).

Optionally, the immunoglobulin toll-like receptor antagonist is an antibody toll-like receptor antagonist or antibody fragment toll-like receptor antagonist. Further optionally, the immunoglobulin toll-like receptor antagonist is a murine antibody toll-like receptor antagonist or murine antibody fragment toll-like receptor antagonist. Still further optionally, the immunoglobulin toll-like receptor antagonist is a humanised antibody toll-like receptor antagonist or humanised antibody fragment toll-like receptor antagonist. Still further optionally, the immunoglobulin toll-like receptor antagonist is a monoclonal antibody toll-like receptor antagonist or monoclonal antibody fragment toll-like receptor antagonist.

Optionally, the immunoglobulin toll-like receptor antagonist is selected from Tomaralimab (“OPN-305” as disclosed in U.S. Pat. No. 8,734,794) and T2.5 (as disclosed in U.S. Pat. No. 8,623,353).

Optionally, the lipid-A analogue toll-like receptor antagonist is OM-174 (as disclosed in WO2006095270).

Optionally, the method comprises administering the toll-like receptor antagonist to the retinal pigment epithelium. Further optionally, the method comprises administering the toll-like receptor antagonist to the plasma membrane of the retinal pigment epithelium. Still further optionally, the method comprises administering the toll-like receptor antagonist apically and/or basolaterally to the plasma membrane of the retinal pigment epithelium.

Optionally, the method comprises administering the toll-like receptor antagonist to the retinal immune cells. Further optionally, the method comprises administering the toll-like receptor antagonist to the plasma membrane of the retinal glia and/or mononuclear phagocytes. Still further optionally, the method comprises administering the toll-like receptor antagonist to the retinal microglia cells, muller glia cells and/or mononuclear phagocytes.

Optionally, the method at least reduces photoreceptor cell death. Further optionally, the method reduces photoreceptor cell death. Still further optionally, the method inhibits photoreceptor cell death.

Optionally, the method at least reduces oxidative-stress-induced photoreceptor cell death. Further optionally, the method reduces oxidative-stress-induced photoreceptor cell death. Still further optionally, the method inhibits oxidative-stress-induced photoreceptor cell death.

Optionally, the method at least reduces photoreceptor cell death in at least one row of photoreceptors. Further optionally, the method reduces photoreceptor cell death in at least one row of photoreceptors. Still further optionally, the method inhibits photoreceptor cell death in at least one row of photoreceptors.

Optionally, the method at least reduces oxidative-stress-induced photoreceptor cell death in at least one row of photoreceptors. Further optionally, the method reduces oxidative-stress-induced photoreceptor cell death in at least one row of photoreceptors. Still further optionally, the method inhibits oxidative-stress-induced photoreceptor cell death in at least one row of photoreceptors.

Optionally, the method at least reduces photoreceptor cell death in at least two rows of photoreceptors. Further optionally, the method reduces photoreceptor cell death in at least two rows of photoreceptors. Still further optionally, the method inhibits photoreceptor cell death in at least two rows of photoreceptors.

Optionally, the method at least reduces oxidative-stress-induced photoreceptor cell death in at least two rows of photoreceptors. Further optionally, the method reduces oxidative-stress-induced photoreceptor cell death in at least two rows of photoreceptors. Still further optionally, the method inhibits oxidative-stress-induced photoreceptor cell death in at least two rows of photoreceptors.

Optionally, the method at least reduces retinal degeneration. Further optionally, the method reduces retinal degeneration. Still further optionally, the method inhibits retinal degeneration.

Optionally, the method at least reduces oxidative-stress-induced retinal degeneration. Further optionally, the method reduces oxidative-stress-induced retinal degeneration. Still further optionally, the method inhibits oxidative-stress-induced retinal degeneration.

Optionally, the method at least reduces retinal pigment epithelium fragmentation. Further optionally, the method reduces retinal pigment epithelium fragmentation. Still further optionally, the method inhibits retinal pigment epithelium fragmentation.

Optionally, the method at least reduces oxidative-stress-induced retinal pigment epithelium fragmentation. Further optionally, the method reduces oxidative-stress-induced retinal pigment epithelium fragmentation. Still further optionally, the method inhibits oxidative-stress-induced retinal pigment epithelium fragmentation.

Optionally, the method comprises parenteral administration of the toll-like receptor antagonist.

Optionally, the method comprises injection of the toll-like receptor antagonist. Further optionally, the method comprises retinal injection of the toll-like receptor antagonist. Still further optionally, the method comprises sub-retinal injection of the toll-like receptor antagonist.

Optionally, the method comprises intravitreal administration of the toll-like receptor antagonist.

Optionally, the method comprises topical administration of the toll-like receptor antagonist. Further optionally, the method comprises topical administration of the toll-like receptor antagonist to one or both eyes. Still further optionally, the method comprises topical administration of the toll-like receptor antagonist to the surface of one or both eyes.

Optionally, the method comprises the further step of decreasing the expression or activation of Myeloid differentiation primary response 88 (MYD88) in the subject.

Optionally or additionally, the method comprises the further step of decreasing the expression or activation of MyD88-adapter-like (Mal) in the subject.

Optionally, the method comprises the further step of decreasing the expression or activation of MyD88 and Mal in the subject.

### BRIEF DESCRIPTION OF THE DRAWINGS

Embodiments of the present invention will be described with reference to the appended non-limiting examples and the accompanying drawings in which:

FIG. 1 illustrates oxidative stress induced TLR2 activation induces AP complement factor expression, wherein qPCR of CFB (a, c) and C3 (b, d) expression in BMDMs or THP1s treated with 20 nM Pam3Cys4 is shown, IHC of C3d (purple) in healthy non-disease donor (e, f) and AMD donor eyes (g-i) is shown, wherein black arrow and black asterisk denote C3d in CC and basal laminar deposits in AMD donor eye (representative of N=4 non-disease donor eyes, N=5 AMD donor eye), wherein CC: choriocapillaris; RPE: retinal pigment epithelium; BM: Bruch's membrane, wherein qPCR of CFB (j) and C3 (k) in ARPE-19 cells treated with 20 nm Pam3Cys4 is shown, wherein data shown are mean±SD for a representative of 3 separate experiments, wherein (1) generation and chemical structure of CEP-adduct from DHA, (m, n) qPCR of C3 and CFB in hfRPE cells treated with 0.1 μg IgG or anti-TLR2 antibody prior to 10 μM CEP-HSA for 24 h, (o) IHC of TLR2 (purple) in a healthy donor are shown, wherein bottom panel (black box) is photo bleached to illustrate apical and basolateral RPE immunoreactivity (N=4 non-disease donor eyes), wherein (p) secreted CFB at 24 h and C3 at 48 h in hfRPE cells treated with 17.5 μM or 35 μM CEP-HSA and 20 nM Pam3Cys4 is shown with mean±SD representative of 3 independent experiments p<0.05*, p<0.01** and p<0.001*** (see also FIGS. 8&9);

FIG. 2 illustrates neutralization of TLR2 in a photo-oxidative model of retinal degeneration and in mononuclear cells decreases C3 expression and deposition, wherein (a-d) 3 μg of anti-TLR2 or anti-IgG was injected IVT into C57Bl6 mice which were then exposed to 100K lux light for 7 days, wherein (a) quantification of photoreceptor cell rows in anti-IgG vs anti-TLR2 groups, (b) IF of C3 (green) and nuclear DAPI (blue) anti-IgG vs anti-TLR2 mice (scale bars: 20 um), (d) quantification of outer retinal C3 positive cells/deposits detected in ONL and subretinal space are shown, wherein data shown are mean±SEM p value was determined by nonparametric t test p<0.05. n=9-10 per experiment, *=p<0.05, wherein (e) BMDMs from WT, TLR2^{-/-}, Mal^{-/-} or MyD88^{-/-} mice were treated for 3, 6 and 24 h with 20 nM Pam3Cys4 is shown, expression of (e) C3 (f) CFB was assayed by RT-PCR, wherein (g, h) HEK293-TLR2 cells were transfected for 24 h with C3 promoter-luciferase (100 ng), Renilla-luciferase (40 ng) and empty vector (EV) or plasmid expressing (g) Mal or (h) MyD88 at 10, 50 and 80 ng, wherein results are normalised for Renilla luciferase activity and represented as relative stimulation over the non-stimulated EV control, mean+/−SD for triplicate determinations p value determined by one-way ANOVA and Tukey post test p<0.05 (denoted *), p<0.01 (**), p<0.001 (***), wherein secreted C3 expression in (i) BMDMs and (j) primary mouse microglia treated with 20 nM Pam3Cys4 for 6 and 24 or 48 h is shown;

FIG. 3 illustrates TLR2^{-/-} mice are protected from oxidative stress induced RPE damage, wherein (a-f) histological H&E analysis of WT and TLR2^{-/-} mice injected IV, via tail vein, with NaCl or NaIO₃ (50 mg/kg), (c, d) quantification of RPE area (in blue)/pixel (scale bars: 20 um), (e) number of RPE breaks/frame, (f) distance between RPE monolayer breaks measured using image j (n>5 mice per genotype), (g) IF of ZO-1 on RPE flatmounts from WT mice and TLR2^{-/-} mice 8 h post NaIO₃ (n>5 per genotype) (asterix: ZO-1 loss, closed arrows: areas cobblestone patterning, open arrows: areas of discontinuous membrane staining), (h) ZO-1 western blot in hfRPE cells treated with 17.5 μM or 35 μM CEP-HSA and 20 nM Pam3Cys4, 24 h are shown (see also FIG. 10-12);

FIG. 4 illustrates TLR2 deficiency protects against NaIO₃-induced photoreceptor cell death and complement factor C3 deposition, wherein WT and TLR2^{-/-} mice were injected IV, with NaCl or NaIO₃ (50 mg/kg) and eyes enucleated 72 h later, wherein (a,b) TUNEL (red) immunoreactivity (scale bars: 20 um) and quantification, (c) number of photoreceptor cell rows in the ONL are shown, wherein data shown are mean±SEM p value determined by nonparametric t test p<0.05. IF of C3 in (d) WT vs (e) TLR2^{-/-} mice, wherein (f-g) expression of C3 cleavage fragments in RPE/choroid tissue from WT and TLR2^{-/-} mice injected IV, with NaCl or NaIO₃ (g) quantification of iC3b is shown;

FIG. 5 illustrates oxidative stress products drive AP activation and MAC formation in a TLR2 dependent manner, wherein (a, b) polarised hfRPE cells or (c-e) ARPE-19 cells were treated with 10% Hi-NHS or NHS in combination with HSA or CEP-HSA for 24 h, phase transmission, presence of MAC (red), Phalloidin (green), DAPI (blue), representative images from 3 separate experiments is shown, wherein (b, e) quantification of MAC+ specks in 3 30×frames, data mean±SD, one-way ANOVA followed by Tukey post-test used to determine significance between groups p<0.001 (***), wherein (f-i) IF of MAC (red), Phalloidin (green), DAPI (blue) in ARPE-19 cells treated with (f, g) 0.1 μg IgG or anti-TLR2 antibody for 1 h or (h,i) with DMSO or 40 μm Mal peptide inhibitor for 2 h, (f-i) prior to CEP-HSA and 10% NHS for 24 h, (g,i) quantification of MAC+ specks in 4 20×frames, mean±SD p value determined by nonparametric t test p<0.05, (j-o) WT and TLR2^{-/-} mice injected IV, with NaIO₃ (50 mg/kg), (j) quantification of MAC fluorescent intensity (Scale bars; 20 um), (k-o) representative IF images of MAC at 72 h are shown, wherein lysed tissue was assayed by western blot for expression of CFB(Bb), C9 and C9b at (p) 24 h and (r) 72, wherein (q, s) mean pixel density for C9b was quantified using image J (see also FIG. 13-14);

FIG. 6 illustrates TLR2 deficiency reduces oxidative stress induced Iba1 positive mononuclear cell infiltration, wherein (a,b) ARPE-19 cells were treated with 10% NHS, CEP-HSA or NHS plus CEP-HSA and (a) LDH activity (b) MCP-1 was assayed, wherein (c) MCP-1 secretion from ARPE-19 cells untreated or treated with anti-TLR2 0.1 μg for 1 h prior to 10% NHS, CEP or both for 24 h, (d-g) WT and TLR2^{-/-} mice were injected IV, with 50 mg/kg NaIO₃ (n>5) are shown, wherein retinal cryosections (d, f) were stained for IBA1 (green) and DAPI (blue) at 72 h (white arrows indicate iba1+ cells subjacent to the RPE), wherein (e) quantification of Iba1+ cells per 20× frame in the ONL and (g) photoreceptor OS are shown, wherein mean±SEM p value determined by nonparametric t test, minimum of 8 frames counted/mouse (n=10), wherein (h) THP-1 monocyte migration (after 120 min) from top chamber (8 μm pore transwells), towards fresh complete media, or conditioned media from RPE cells that were untreated, treated with CEP for 24 h, or treated with CEP+NHS, mean+/−SEM for 4-5 counts *p<0.05. (i) MCP_1 secreted from ARPE-19 cells treated with CEP-HSA or CEP-HSA in the presence of NHS for 24 h. (j) CD86 MFI in CD45+CD66b−CD14+CD16+ monocytes treated with MCP-1 for 24 h are shown, wherein experiments were carried out in triplicate and data are mean±SEM for 3 separate experiments, wherein the p value was determined by nonparametric t test, wherein (k) polarized ARPE-19 cells treated with NHS, CEP or both NHS and CEP were assayed for secretion of MCP-1 by ELISA in both apical and basolateral supernatants, wherein all images scale bars=20 um;

FIG. 7 illustrates pharmacological blockade of TLR2 rescues NaIO₃-induced RPE and photoreceptor cell degeneration, wherein (a-c) wildtype mice were injected IV with NaCl or NaIO₃ (50 mg/kg) (n=4 per treatment), wherein, at the same time, mice were injected IVT with 3 μg of anti-TLR2 antibody or anti-IgG, wherein 72 h later eyes were prepared for (a, top row) H&E and (a, bottom row) TUNEL analysis, wherein (b) quantification of TUNEL+ cells in NaIO₃ injected WT mice in combination with anti-IgG vs anti-TLR2 and (c) number of photoreceptor cell rows in IgG injected vs anti-TLR2 ab injected mice following NaIO₃ treatment are shown, wherein scale bars=20 um;

FIG. 8 illustrates pattern of deposition of C3d differs in non-disease and AMD donor eyes, wherein immunohistochemistry of C3d in (a) healthy non-disease donors and (b) AMD donor eyes are shown, wherein immunoreactivity is visible in the choriocapillaris (CC) of both healthy and disease eyes and sub-RPE in basal laminar deposits (BLD) and drusen in the AMD donor eyes, wherein immunoreactivity is also visible in linear deposits (black arrow*) and in migrating cells (white arrows) wherein CC: choriocapillaris; RPE: retinal pigment epithelium; BM: Bruch membrane;

FIG. 9 illustrates TLR2 activation of RPE weakly induces Cl and CS but not MASPl or C4, wherein (a-d) ARPE-19 cells were treated with 20 nm Pam3Cys4 over 24 h, complement factors Cl, MASP1, C5 and C4 were assayed by RT-PCR, wherein experiments were carried out in triplicate and data are mean±SEM for 3 separate experiments;

FIG. 10 illustrates Nal03 induces TLR2, CFB and C3 expression in ARPE-19 cells, wherein (a-c) ARPE-19 cells were treated with 1, 5 or 10 mM NaI03 for 24 h expression of (a) TLR2, (b) CFB and (c) C3 were assayed by quantitative RT-PCR;

FIG. 11 illustrates the Nal03 model of retinal degeneration generates CEP-adducts, wherein wildtype C57Bl6 mice were injected intravenously (IV), via tail vein, with either saline (NaCl) or NaI03 (50 mg/kg), wherein eyes were enucleated 24 h later and prepared for histological analysis of CEP (brown DAB stain), wherein immunoreactivity is visible in the ONL in mice injected with NaI03 and absent in mice injected with NaCl when compare to an IgG control;

FIG. 12 illustrates the alternative complement pathway causes RPE blebbing and photoreceptor cell death in the Nal03 model of retinal degeneration, wherein (a) H&E stain of wildtype C57Bl6 mice (n=4) injected intravenously (IV) via tail vein with saline (NaCl), eyes were enucleated 72 h later and prepared for immunohistochemistry, (b-f) Wildtype C57Bl6 mice (n=4) were injected intravenously (IV) via tail vein with NaI03; at the same time, mice were injected intravitreally (IVT) with anti-CPD antibody or anti-IgG. Eyes were enucleated 72 h later and prepared for immunohistochemistry: (b,c) top panels; H&E stained, bottom panels; red=TUNEL, blue=DAPI, (d) quantification of the number of pigmented cells in the photoreceptor outer segments, (e) quantification of the number of photoreceptor ONL rows, (f) the number of TUNEL positive cells in the ONL per frame are show, wherein data shown are mean±SEM p value was determined by nonparametric t test, p<0.01 (**);

FIG. 13 illustrates (a, b) polarised htRPE cells or (c-e) ARPE-19 cells were treated with 10% Heat inactivated (Hi) normal human serum (NHS) or NHS in combination with human serum albumin (HSA) or CEP-HSA for 24 h Soluble MAC production was quantified in the cell culture supernatants by ELISA; and

FIG. 14 illustrates oxidative stress induced MAC formation is blocked in CS deficient mice, wherein wildtype C57Bl6 and DBA/2J (C5 KO) mice were injected intravenously (IV), via tail vein, with Nal03 (50 mg/kg), eyes were enucleated at 72 h and prepared for MAC immunofluorescence and H&E stain, wherein (f) quantification of ONL is shown.

### EXAMPLES

### Materials and Methods

### In Vivo Animal Studies

### NaIO₃ Model of Retinal Degeneration

All experiments were conducted in accordance with the ARVO Statement for Use of Animals in Ophthalmic and Vision Research, and approved by the Trinity College Dublin Animal Research Ethics Committee or the Australian National University (ANU) Animal Experimentation Ethics Committee. Mice used were C57BL/6J mice and Tlr2^{-/-} (JAX stock #004650) at 8-12 week old. DBA/2J CS-deficient mice (JAX stock #000671) were 10 months old and matched to 10 month old C57BL/6J mice. Comparator WT vs KO mice were sex matched, as sex has a measurable effect on the NaIO₃ model.

### RPE Flatmounts

A single intravenous injection, via tail vein, of NaIO₃ (50 mg/kg) was administered to C57Bl/6J and TLR2^{-/-} mice. Mice were euthanized 8 hours post injection, eyes fixed in ice-cold methanol for 15 minutes and the choroid/RPE dissected into flatmounts. Flatmounts were blocked and permeabilised in 5% NGS, 0.05% Triton X100 for 1 hour and incubated with ZO-1 (1:100, Invitrogen) overnight at 4° C. Flatmounts were washed with PBS and incubated with goat anti-rabbit 488 for 2 hours at room temperature.

### Iba1, C3 and MAC Staining

A single intravenous injection, via tail vein, of NaIO₃ (50 mg/kg) was administered to C57Bl/6J and TLR2^{-/-} mice. Mice were euthanized 72 hours post injection, eyes fixed in 4% paraformaldehyde for 90 minutes and after a 30% sucrose gradient, eyes were embedded in optimal cutting temperature compound (OCT). 12 um sections were blocked and permeabilised in 5% NGS, 0.05% Triton X100 for 1 hour and incubated with C3 (Abcam ab11887, 1:100), MAC (Biozol, FGI-10-1801, 1:100) or Iba1 (Wako 019-19741, 1:500) overnight at 4° C. Sections were washed with PBS and incubated with Alexa Fluor® goat anti-rabbit 488 or Alexa Fluor® goat anti-mouse 488 1:500 in 5% NGS for 2 hours at room temperature and counterstained with Hoechst. To quantify the numbers of Iba1+ cells, a minimum of eight 20× objective frames were counted per eye and counts were averaged per mouse. Images were cropped to only include the RPE and photoreceptor layers for MAC staining and the mean fluorescence intensity was measured.

### CEP Staining

A single intravenous injection, via tail vein, of NaIO₃ (50 mg/kg) was administered to C57Bl/6J mice. Mice were euthanized 24 hours post injection, eyes fixed in 4% paraformaldehyde for 90 minutes and after a 30% sucrose gradient, eyes were embedded in OCT. 12 um sections were stained using anti-CEP ab 1:1000 (Kindly provided by Sheldon Rowan, Tufts University, USA) using the Vectastain ABC Kit following manufacturers protocol and detected using DAB (Vector Laboratories).

### Inhibition by Anti-CFD Blocking Antibody

A single intravenous injection was administered via the tail of NaIO₃ (50 mg/kg) in NaCl. Control mice received NaCl. In tandem, mice received a single subretinal injection of either anti-CFD antibody (R&D) antibody or IgG control (0.5 μg per eye). Mice were euthanized 72 hours post injection. Eyes were fixed in Davidson's fixative overnight, washed 3 times in PBS and embedded in paraffin wax. 5 μm sections were cut with a microtome (Leica) and subject to xylene deparaffinising and ethanol rehydration. For histology slides were stained with haematoxylin and eosin. To detect cell death sections were stained using in situ Cell Death Detection kit, TMR red (Roche) following manufacturer's protocol and nuclei counterstained with Hoechst.

### Inhibition by Anti-TLR2 Blocking Antibody

A single intravenous injection was administered via the tail of NaIO₃ (50 mg/kg) in NaCl. Control mice received NaCl. In tandem, mice received a single subretinal injection of either anti-TLR2 blocking (Invivogen) antibody or IgG control (3 μg per eye). Mice were euthanized 3 days' post injection eyes were fixed in Davidson's fixative overnight, washed 3 times in PBS and embedded in paraffin wax. 5 μm sections were cut with a microtome (Leica) and subject to xylene deparaffinising and ethanol rehydration. For H&E histology slides were stained with haematoxylin and eosin. TUNEL staining—To detect cell death sections were stained using in situ Cell Death Detection kit, TMR red (Roche) for 1 hour at 37° C. following manufacturer's protocol and nuclei counterstained with Hoechst. The number of photoreceptor rows was calculated by counting the number of nuclei spanning the height of the outer nuclear layer (ONL) at three individual points per 20× frame (eg. ˜12 nuclei height in ONL of wildtype mice) and an average was taken per 20× frame. To quantify the numbers of ONL rows or TUNEL+ cells, a minimum of eight 20× objective frames were counted per eye and counts were averaged per mouse.

### Photo-Oxidative Stress Model of Retinal Degeneration

C57BL/6J mice (8 weeks old) received a single intravitreal injection of either an anti-TLR2 antibody or IgG control (3 μg per eye). Animals were then exposed to 100 Klux light for 7 days to induce photo-oxidative damage, as described previously (NATOLI, R., JIAO, H., BARNETT, N. L., FERNANDO, N., VALTER, K., PROVIS, J. M. & RUTAR, M. 2016a. A model of progressive photo-oxidative degeneration and inflammation in the pigmented C57BL/6J mouse retina. Exp Eye Res, 147, 114-27). Following photo-oxidative damage, animals were euthanized and eyes collected for histological analysis. Retinal cryosections were stained with TUNEL (Roche) to detect photoreceptor cell death. C3 immunohistochemistry was performed using α-C3 antibody (1:100, Abcam), and C3+ cells/deposits in the outer retina (between the ONL and RPE) were counted per retinal section.

### Human Studies

### Immunohistochemistry in Human Tissue

Human donor eyes obtained from the Iowa Lions Eye Bank (Iowa City, Iowa, USA) eyes were processed within 8 hours of death (Table 1). Macular punches which had been fixed in 4% paraformaldehyde and embedded in sucrose-optimal cutting medium were sectioned on a cryostat and stained for TLR2 (Abcam) and C3d (Dako) using VIP and Vectastain ABC Kit (Vector Laboratories). Characteristics of donor tissue used for immunohistochemistry are displayed in Table 2.

TABLE 1
Key Resources Table

| | | |
|---|---|---|
| REAGENT or RESOURCE | SOURCE | IDENTIFIER |

Antibodies

| | | |
|---|---|---|
| Anti-TLR2 | Abcam | Cat# ab1655; RRID: AB_302428 |
| Anti-TLR2 neutralizing | InvivoGen | Clone T2.5; Cat# mab-mtlr2; RRID: |
| | | AB_763722 |
| Mouse Control IgG1 | InvivoGen | Cat# mabg1-ctrlm; RRID: AB_11203233 |
| Anti-C3 | MP-Biomedicals | Cat# 855444 |
| Anti-C3 | Abcam | Cat# ab11887; RRID: AB_298669 |
| Anti-C3d | Agilent | Cat# A006302; RRID: AB_578478 |
| Anti-CFB | Atlas Antibodies; Sigma | Cat# HPA001817; RRID: AB_1078779 |
| Anti-CFD | R&D systems | Cat# MAB5430; RRID: AB_10640506 |
| Rat IgG1 Isotype Control | R&D systems | Cat# MAB005; RRID: AB_357348 |
| MAC/anti-C5b-9 for Mouse | Biozol | Cat# FGI-10-1801 |
| IHC | | |
| MAC/anti-C5b-9 for human | Santa Cruz Biotechnology | clone aE11; Cat# sc58935; RRID: |
| IHC | | AB_1119839 |
| MAC/anti-C5b-9 for WB | Santa Cruz Biotechnology | Clone 2A1; Cat# sc66190; RRID: |
| | | AB_1119840 |
| Anti-CEP | Dr. Rowan (Rowan et al., | NA |
| | 2017) | |
| ZO-1 | Thermo Fisher Scientific | Cat# 40-2200; RRID: AB_2533456 |
| IBA1 | Wako | Cat# 019-19741; RRID: AB_839504 |
| Biotinylated Goat Anti-Rabbit | Vector Laboratories | Cat# BA-1000; RRID: AB_2313606 |
| IgG | | |
| Biotinylated Rabbit Anti-Goat | Vector Laboratories | Cat# BA-5000; RRID: AB_2336126 |
| IgG | | |
| Anti-human CD16 | BioLegend | Clone 3G8; Cat# 302006; RRID: |
| | | AB_314206 |
| Anti-human CD86 | Miltenyi Biotec | Clone FM95; Cat# 130-094-877; RRID: |
| | | AB_10839702 |
| Anti-human CD45 | BioLegend | Clone 2D1; Cat# 368506; RRID: |
| | | AB_2566358 |
| Anti-CD66b | BioLegend | Clone G10F5; Cat# 305116; RRID: |
| | | AB_2566605 |
| Anti-human CD14 | Miltenyi | Clone Tük4; Cat# 130-098-058; RRID: |
| | | AB_2660173 |
| Anti-human CD80 | Miltenyi | Clone 2D10; Cat# 130-099-710; RRID: |
| | | AB_2659260 |
| Alexa Fluor 488 Phalloidin | Thermo Fisher Scientific | Cat# A12379; RRID: AB_2315147 |
| Alexa Fluor 647 Donkey Anti- | Abcam | Cat# AB150107 |
| Mouse | | |
| Alexa Fluor 488 Goat Anti- | Thermo Fisher Scientific | Cat #A11034; RRID: AB_2576217 |
| Rabbit | | |
| Alexa Fluor 488 Goat anti- | Thermo Fisher Scientific | Cat #A11001; RRID: AB_2534069 |
| Mouse | | |

Biological Samples

| | | |
|---|---|---|
| Human donor eye tissue | The Iowa Lions Eye Bank | https://iowalionseyebank.org/ |
| Peripheral Blood Mononuclear | Isolated from healthy | NA |
| Cells (PBMCs) | volunteers | |
| Primary Microglia | Isolated as per Fernando et al., | NA |
| | 2016 | |
| Normal Human Serum | Sigma | Cat# H4522-20ml |

Chemicals, Peptides, and Recombinant Proteins

| | | |
|---|---|---|
| Mouse M-CSF | Miltenyl Biotec | Cat# 130-094-129 |
| Mouse Recombinant GM-CSF | Stem Cell Technologies | Cat# 78017.1 |
| Pam3Cys4 | Invivogen | Cat# tlrl-pms |
| CEP-HSA | Prof. Salomon (Gu et al., 2003) | |
| Mal (TIRAP) Inhibitor Peptide | Calbiochem | Cat# 613570-1MG |
| Sodium Iodate | Sigma | Cat # S4007-100G |
| Hoechst | Sigma | Cat #B2261-25MG |
| Collagen IV | Sigma | Cat# C5533-5MG |

Critical Commercial Assays

| | | |
|---|---|---|
| In SITU Cell Death Detection | Roche | Cat# 12156792910 |
| Kit, TMR red | | |
| MCP-1 ELISA | Tebubio | Cat# 900-K31 |
| MAC ELISA | Abbexa | Cat# abx350654 |
| Isolate II RNA Extraction Kit | Bioline | Cat# BIO-52073 |
| MMLV Reverse Transcriptase | Promega | Cat# M1705 |
| SensiFast SYBER Green | Bioline | Cat# BIO-92020 |
| Pierce LDH Viability Assay | Thermo Fisher Scientific | Cat# 13464269 |
| LIVE/DEAD Aqua | Thermo Fisher Scientific | Cat# L34957 |
| Mouse Vectastain ABC HRP | Vector Laboratories | Cat#PK-4002; RRID: AB_2336811 |
| Kit | | |
| ImmPACT DAB Substrate | Vector Laboratories | Cat#SK-4105; RRID: AB_2336520 |
| Vectastain Elite ABC HRP kit | Vector Laboratories | Cat#PK-6200; RRID: AB_2336826 |
| Vector VIP Peroxidase (HRP) | Vector Laboratories | Cat# SK-4600; RRID: AB_2336848 |
| Substrate Kit | | |

Experimental Models: Cell Lines

| | | |
|---|---|---|
| ARPE19 | ATCC | CRL-2302 |
| Immortalized BMDMS | Prof. Golenbock, UMASS, | N/A |
| | USA | |
| hfRPE | Dr. A. Maminishkis | N/A |
| | (Maminishkis et al., 2006) | |
| THP-1 | ATCC | TIB-202 |
| HEK293-TLR2 | Dr. K. Fitzgerald, UMASS, | N/A |
| | USA | |

Experimental Models: Organisms/Strains

| | | |
|---|---|---|
| C57B16 | Jackson labs | |
| Tlr2−/− | Jackson labs | JAX stock #004650 |
| DBA/2J | Jackson labs | JAX stock #000671 |

Oligonucleotides

| | | |
|---|---|---|
| Primers for SYBER qPCR, | This disclosure | N/A |

Recombinant DNA

| | |
|---|---|
| C3 luciferase (T1 del) | Addgene |

Software and Algorithms

| | | |
|---|---|---|
| GraphPad Prism | GraphPad Software | https://www.graphpad.com |
| ImageJ | | https://imagej.nih.gov/ij/ |
| FlowJo | Tree star | https://www.flowjo.com |

TABLE 2
Characteristics of Donor Tissue used for Immunohistochemistry
Related to STAR Methods.

| | | | | | Death- |
|---|---|---|---|---|---|
| | Disease | Age | | | Processing |
| I.D. | Stage | (years) | Gender | Cause of death | (time) |
| CTL 1 | Control | 87 | Male | Cancer | 02:57 |
| CTL 2 | Control | 91 | Female | Unavailable | 03:08 |
| CTL 3 | Control | 90 | Female | Carcinoma | 05:40 |
| CTL 4 | Control | 79 | Male | Unavailable | 05:32 |
| AMD 1 | Early AMD | 99 | Female | Acute Pancreatitis | 04:49 |
| AMD 2 | Early AMD | 89 | Female | Unavailable | 03:54 |
| AMD 3 | Early AMD | 89 | Female | Ischemic | 06:46 |
| | | | | Cardiomyopathy | |
| AMD 4 | Early AMD | 95 | Female | Respiratory | 05:59 |
| | | | | Failure, COPD | |
| AMD 5 | Early AMD | 79 | Female | COPD | 05:55 |
| AMD 6 | Early AMD | 91 | Male | Aspiration | 07:48 |
| | | | | Pneumonia | |

### Cell Lines

ARPE-19 cells (ATCC CRL 2302) 1:1 mixture of Dulbecco's modified Eagle's medium (DMEM)/nutrient mixture F-12 Ham with L-glutamine, 15 mM HEPES, sodium bicarbonate. THP-1 cells RPMI 1640 medium Immortalized bone marrow derived macrophages wildtype MyD88^{-/-} and Mal^{-/-} mice (Kind gift Prof. Golenbock, UMass Medical School) DMEM. Peripheral blood mononuclear cells (PBMCs) were isolated from human blood RPMI 1640. All medium was supplemented with 10% fetal bovine serum (FBS) and 1% Penicillin-Streptomycin (Sigma-Aldrich). YFP+Cx3cr1-expressing microglia were isolated from mouse retinas according to previously described methods (FERNANDO, N., NATOLI, R., VALTER, K., PROVIS, J. & RUTAR, M. 2016. The broad-spectrum chemokine inhibitor NR58-3.14.3 modulates macrophage-mediated inflammation in the diseased retina. J Neuroinflammation, 13, 47) and were sorted into a 48-well plate at 1500 cells per well. Isolated primary microglia were cultured for 3 weeks in DMEM-F12 supplemented with 10% FBS, 1% antibiotic-antimycotic (Thermo Fisher Scientific), 3% L-glutamine, 0.25 ng/ml GM-CSF (Stem Cell Technologies) and 2.5 ng/ml M-CSF (Miltenyi Biotec) prior to TLR2 stimulation. Cells were maintained at 37° C., 5% CO₂, 95% air.

### Primary Human Fetal RPE Culture

Cells, provided by Dr. Arvydas Maminishkis from the National Eye Institute (NEI), Bethesda, USA, were received as a confluent monolayer of P-0 cells assays were conducted using cells at P-1. Primary human fetal RPE cells were isolated from human donor eyes as previously described and cultured in MEM-α containing 5% FCS (MAMINISHKIS, A., CHEN, S., JALICKEE, S., BANZON, T., SHI, G., WANG, F. E., EHALT, T., HAMMER, J. A. & MILLER, S. S. 2006. Confluent monolayers of cultured human fetal retinal pigment epithelium exhibit morphology and physiology of native tissue. Invest Ophthalmol Vis Sci, 47, 3612-24).

### Method Details

### Stimulation of Cells

Bone marrow derived macrophages (BMDMs), human monocytic cell like THP1s, PBMCs ARPE-19 cells, primary human fetal RPE cells or primary retinal microglia were stimulated with the generic TLR2/1 ligand Pam3Cys4 (Invivogen) or with CEP-HSA (kindly provided by Prof. Robert G Salomon (Case Western Reserve University, Cleveland Ohio) at indicated concentrations. Where indicated RPE cells were pre-treated with 0.1 μg/ml TLR2 antibody for 1 hour (T2.5 Invivogen), corresponding IgG control 0.1 μg/ml (Invivogen), Mal peptide inhibitor resuspended in DMSO 40 μM (Calbiochem) or an equal volume of DMSO was used in the control treatment.

### Polarised ARPE-19 Cell Culture

0.4 μM polyester transwell inserts (VWR) were coated with 100 μg/ml Collagen IV (Sigma-Aldrich C5533) for 4 hours. ARPE-19 cells were seeded at a density of 1.7×10⁵ cells per cm² in DMEM F-12 Ham containing 10% (FBS). Two days later medium was replaced with complete medium containing 1% FBS and replenished twice weekly for 4-6 weeks.

### Western Blot

Antibodies for CFB (Atlas Antibodies Sigma) 1:250, C3 (MP Biomedicals-855444), C3d 1:1000 (Dako), ZO-1 (Invitrogen) 1:1000 and C5b-9 (Santa Cruz) 1:500 were incubated overnight at 4° C. Polyvinylidene fluoride (PVDF) membranes were wash 3 times with TBS-T and incubated with horseradish peroxidase conjugated anti-rabbit, anti-mouse or anti-goat 1:2000 (Sigma-Aldrich) for 1 hour at room temperature and developed using enhanced chemiluminescence. Densitometry was used to determine relative quantity of MAC and iC3b protein relative to actin loading control using Image J software. Scanned images were converted to 8-bit images. Each protein band was measured to obtain the area and mean value. The area was multiplied by the mean to obtain a measurement for each lane. The value obtained for the protein of interest was divided by the value obtained for the actin loading control for the corresponding well.

### Quantitative RT-PCR

Total RNA was extracted from BMDMs, THP1s, ARPE-19 or hfRPE cells using Isolate II RNA extraction kit (Bioline) as per manufacturer's instructions. RNA was reverse transcribed using MMLV Reverse Transcriptase (Promega). Target genes were amplified by real time PCR with SensiFast SYBR Green (Bioline) using the ABI 7900HT system (Applied Biosystems). The cycling threshold method was used for relative quantification after normalisation to the ‘housekeeping’ gene βActin. The primers used were:

Human C3
forward 5′-CTGCCCAGTTTCGAGGTCAT-3′;
reverse 5′-CAATCGGAATGCGCTTGAGG-3′
Human CFB
forward 5′-CAGGAAGGTGGCTCTTGGAG-3′;
reverse 5′-CCCATCCTCAGCATCGACTC-3′
Human TLR2
forward 5′-TGTAGCAACTGGCTTAGTTCA-3′;
reverse 5′-TGGCCACAGAGGAGTCTCTTA-3′
βActin
forward 5′-CGCGAGAAGATGACCCAGATC-3′;
reverse 5′-GAGGCGTACAGGGATAGCAC-3′
Mouse C3
forward 5′-AAGCATCAACACACCCAACA-3′;
reverse 5′-CTTGAGCTCCATTCGTGACA-3′
Mouse CFB
forward 5′-ATAGGCCCATCTGTCTCCCC-3′;
reverse 5′-CAGGTGGCTGTCTGAGGAA-3′

### Measurement of MCP-1/CCL2 and MAC by ELISA

MCP-1 (TebuBio), and Soluble MAC (Abbexa) was detected in cell supernatants by sandwich ELISA according to manufacturer's instructions. Absorbance was read at 450 nM on a 96 well plate spectrophotometer.

### Measurement of Membrane Attack Complex In Vitro

Polarised ARPE-19 cells grown on transwell filters were maintained in serum free DMEM F-12 Ham for 48 hours. Cells were stimulated with 10% Normal human serum or heat inactivated normal human serum (Hi) (56° C. 30 minutes) either alone or with human serum albumin (HSA) or CEP-HSA for 24 hours. Where indicated cells were pre-treated for 1 hour with anti-TLR2 blocking antibody (T2.5 Invivogen), IgG control (0.1 μg/ml) or Mal peptide inhibitor (Calbiochem). Supernatants were harvested and assessed for soluble MAC formation by ELISA (Abbexa). Transwell inserts were fixed with 4% Paraformaldehyde for 10 minutes at room temperature, blocked with 5% bovine serum albumin (BSA) for 1 hour at room temperature and incubated with anti-mouse-05b-9 1:25 (Santa cruz) overnight at 4° C. Transwells were washed 3 times in PBS and incubated with goat anti-mouse 647 1:500 (Invitrogen) and Phallodin 1:500 (Invitrogen) for 2 hours at room temperature. Cells were counted stained with Hoechst. Transwells inserts were carefully cut with a sterile blade and mounted on to polysine coated slides (Thermo Scientific) using Mowiol® 4-88. Staining was analysed using a confocal laser scanning microscope Axio Observer Z1 inverted microscope equipped with a Zeiss LSM 700 T-PMT scanning unit and a 40× plan.

### LDH Viability Assay

An LDH cytotoxicity kit (Pierce) was used to detect cell death following MAC formation as per manufacturer's instructions absorbance was read at 490 nm and background absorbance at 680 nm.

### Luciferase Assay

HEK293-TLR2 cells were transfected for 24 hours with C3 promoter-luciferase (100 ng), Renilla-luciferase (40 ng) and empty vector (EV) or plasmid expressing Mal or MyD88 in increasing doses (10, 50 and 80 ng). Results are normalised for Renilla luciferase activity and represented as relative stimulation over the non-stimulated EV control and are expressed as mean+/−SD for triplicate measurements.

### Measurement of Surface ICAM1 and CD86

PBMCs were labelled for the investigation of monocytes with the following fluorochrome-labelled antibodies: anti-CD16 (3G8) anti-CD86 (FM95) anti-CD45 (2D1); anti-CD66b (G10F5); CD14 (Tuk4); CD80 (2D10); (Biolegend or Miltenyi). Each staining well contained 4×10⁵ cells; cells were stained with LIVE/DEAD Aqua (Molecular Probes) followed by staining for 20 min on ice, washed, and analyzed by flow cytometry immediately. Flow cytometry was carried out on a BD LSR Fortessa cell analyzer and analyzed using FlowJo software (Tree Star).

### Quantification and Statistical Analysis

Statistical analysis was carried out using Prism Graphpad and details of each test used can be found in the figure legends.

### Data and Code Availability

This study did not generate/analyse datasets/code Example 1.

### TLR2 Activation Induces AP Complement Factor Expression in Monocytes, Macrophages and the RPE

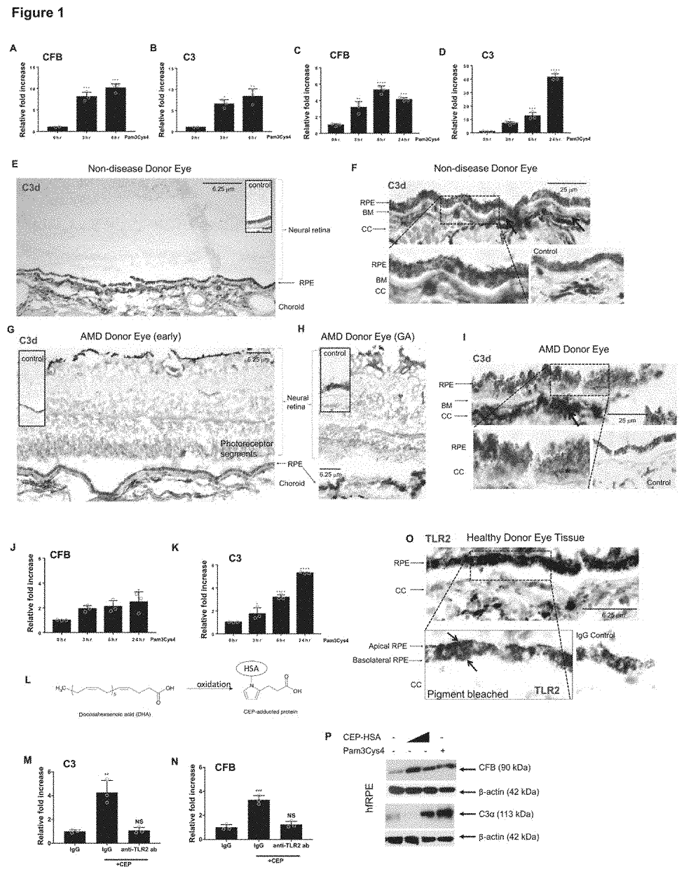

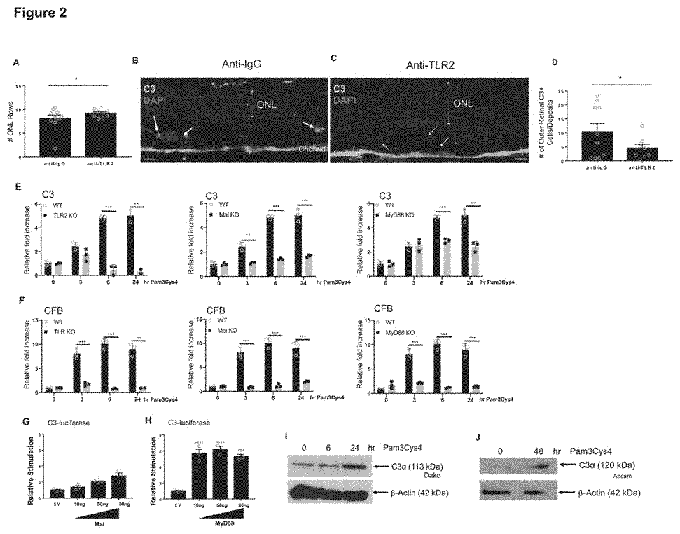

To confirm a role for TLR2 in initiating the complement cascade, gene expression levels of key AP components Complement Factor B (CFB) and Complement factor 3 (C3) in response to TLR2 activation over time with generic TLR2 ligand, Pam3Cys4, a synthetic triacylated lipopeptide PAMP, were measured. Upregulation of CFB and C3 in TLR2 activated bone marrow derived macrophages (BMDMs) (FIG. 1a, b) and human monocytic cell-line (FIG. 1c, d) was observed. Complement production and activation in the retina is reported to be distinct to systemic complement due to the physical barrier provided by Bruch's membrane (BM) basolateral to the retinal pigment epithelium (RPE). In support of this, a clear distinction in C3d staining (purple) in healthy aged donor eyes between the neural retina and the choroid, with the RPE acting as a border (FIG. 1e), was observed. C3d is the final cleavage product of C3 and acts as an opsonin to mark dead and dying cells, or debris to be cleared by innate phagocytes. As C3d is relatively stable, it indicates historical complement activation. Higher magnifications show BM provided a distinct barrier separating the RPE and neural retina from systemic C3d staining in the choroid in healthy donor eyes (FIG. 1f and FIGS. 8&9, representative of N=4). In these non-diseased donor eyes, C3d staining in the blood vessels of the choroid, but no C3d staining adjacent to the RPE/retina, was observed. In contrast, C3d staining in the neural retina and in particular in photoreceptor segments in AMD eyes in both early (FIG. 1g) and late (FIG. 1h) disease was observed. In contrast to healthy donor eyes, C3d staining in AMD donor eyes was apparent immediately subjacent to the RPE both in focused areas, and in a linear pattern immediately below the RPE (FIG. 1i and FIGS. 8&9, representative of N=6). The different patterns of C3d staining between non-disease and AMD donor eyes suggests that either BM can no longer act effectively as a barrier between the outer retina and systemic factors and/or that local cells in the retina can produce complement. It was next assessed whether the RPE itself could respond to TLR2 activation and induce complement. CFB and C3 were significantly up-regulated in RPE cells in response to TLR2 activation (FIG. 1j, k). By comparison there were minor changes in several other complement proteins (see supplementary FIG. 2).

### Example 2

AMD-Associated Oxidative Stress Products Induce AP Complement Secretion from hfRPE Cells

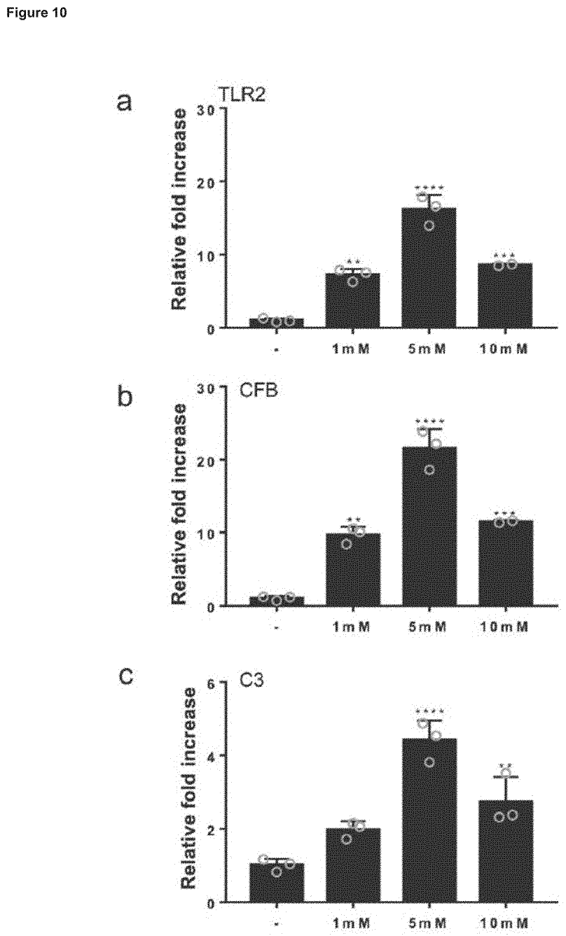

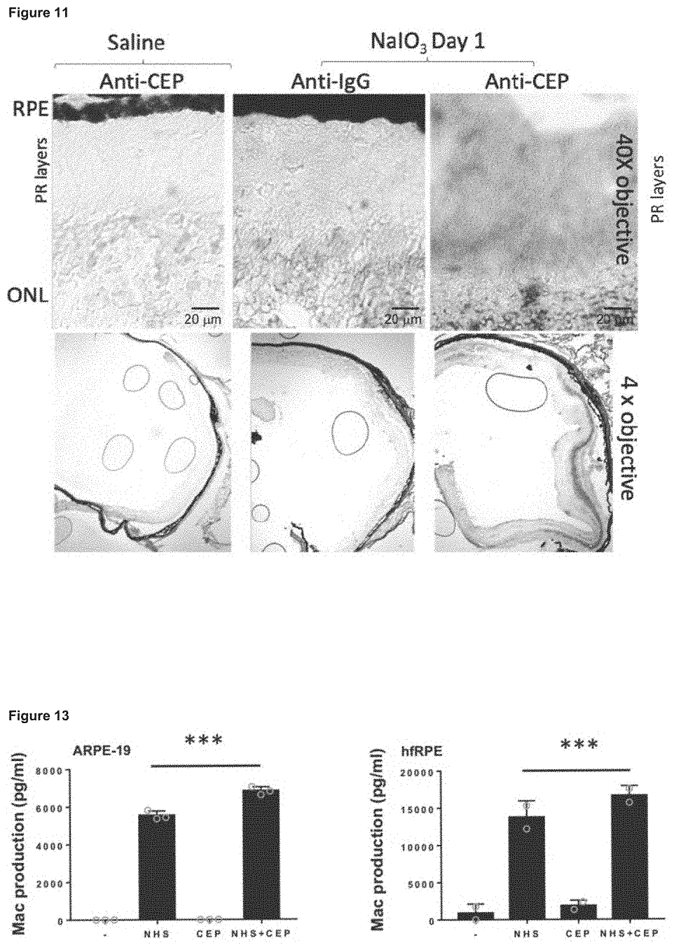

It was next assessed whether a physiologically relevant DAMP generated by oxidative stress could induce the same response in primary human fetal RPE (hfRPE) cells. The retina is one of the most highly metabolically active tissues in the body. This oxidative burden, results in generation of lipid oxidation products such as CEP (FIG. 11). hfRPE cells were incubated with either a neutralizing antibody targeting TLR2 (anti-TLR2) or an IgG control prior to stimulation with CEP-adducted to human serum albumin (CEP-HSA). CEP-HSA induced C3 and CFB transcripts to similar levels observed for Pam3Cys4 in RPE cells and this was inhibited by the presence of anti-TLR2 neutralizing antibodies (FIG. 1m, n). TLR2 localization was assayed in human donor eye tissue and observed both apically and basolaterally in the plasma membrane of the RPE (FIG. 10) Immunoblot analysis demonstrated TLR2 effect on transcript resulted in a change at protein levels with secretion of both CFB and C3 protein into the supernatant of hfRPE cells in response to CEP-HSA or Pam3Cys4 (FIG. 1p). Collectively, these data suggest that cells in the retina have the capacity to generate AP complement factors locally in response to TLR2 activation and oxidative stress product CEP.

### Example 3

### Neutralization of TLR2 in a Photo-Oxidative Stress Model of Retinal Degeneration Decreases C3 Deposition and Promotes Survival of Photoreceptor Cells

Overexpressing C3 in the retina can promote many features of AMD, while inhibiting various complement factors can protect against photoreceptor cell death in models of retinal degeneration. To define a role for TLR2 in bridging oxidative stress to complement activation and assessing its function in retinal degeneration, a well-characterized light-induced photo-oxidative stress model of retinal degeneration was utilized, in which locally produced C3 is known to contribute causally to retinal degeneration. In this model, there is a significant increase in C3+ macrophage/microglia in the photoreceptor layer and a decrease in outer nuclear layer (ONL) thickness. The ONL is made up of the nuclei of the rod and cone photoreceptors, and a decrease in the ONL thickness is indicative of photoreceptor cell death and retinal degeneration. An anti-TLR2 neutralizing antibody or control anti-IgG was injected intravitreally (IVT) into both eyes of each animal. Animals were subsequently exposed to 100K lux light for 7 days continuously. 1-2 more photoreceptor cell rows present in TLR2-neutralised retinas were observed compared to IgG controls, indicating that TLR2 blockade confers protection from oxidative stress induced photoreceptor cell loss in this model (FIG. 2a, b, c). The average number of ONL rows in a healthy wildtype C57Bl/6 retina at the meridian analyzed is ˜12, so a protection of 1-2 rows represents a meaningful preservation of photoreceptor numbers. C3 Immunohistochemistry (IHC) revealed that there was a significant reduction in number of outer retinal C3+ cells/deposits observed in the outer segment (OS) layer of the photoreceptors in anti-TLR2-injected mice compared to IgG controls following photo-oxidative damage (FIG. 2b, c, d) despite the fact that DAPI+ cells are observed in the OS in the anti-TLR2 treated mice as well (FIG. 2c, open arrows). In this model, C3 is deposited by macrophage/microglia that infiltrate the outer retina, therefore, these data indicate that, in vivo, TLR2 signaling in the retina is capable of inducing complement from macrophages/microglia in response to DAMPs generated by photo-oxidative stress, and that loss of TLR2 signaling reduces C3 deposition in the photoreceptor cells enabling photoreceptor cell survival. Inhibition of Mal or MyD88 normally attenuates TLR2-dependent signaling. BMDMs deficient in TLR2 signaling pathway components TLR2, Mal and MyD88 were utilized and AP induction in response to TLR2 activation was measured. An inhibition of C3 (FIG. 2e) and CFB (FIG. 2f) induction was observed in response to TLR2 ligation in TLR2, Mal and MyD88-deficient BMDMs. Conversely, overexpression of Mal or MyD88 through transient transfection leads to a dose-dependent activation of the C3-promoter in luciferase reporter assays (FIG. 2g, h). Finally, to link back to the observations in the photo-oxidative stress model, it was demonstrated that TLR2 activation of BMDMs and primary microglia (MG) isolated from mouse retina induced C3 secretion in response to TLR2 activation (FIG. 2i, j). These data support a model where in response to light induced photo-oxidative damage, endogenous DAMPs activate TLR2 on macrophages/microglia driving gene induction of AP factors and C3 deposition in the outer retina resulting in photoreceptor cell degeneration, confirming a role for TLR2 in bridging oxidative stress to complement deposition in a photo-oxidative stress model. A key feature of the photo-oxidative stress model of retinal degeneration is the primary cell types responding are the mononuclear phagocytic cells. Studies on the pharmacological NaIO₃ model of retinal degeneration have demonstrated the primary initial cell type to respond is the RPE cell.

### Example 4

### Inhibiting Amplification of the AP Ameliorates RPE Degeneration in the NaIO₃ Model of Retinal Degeneration

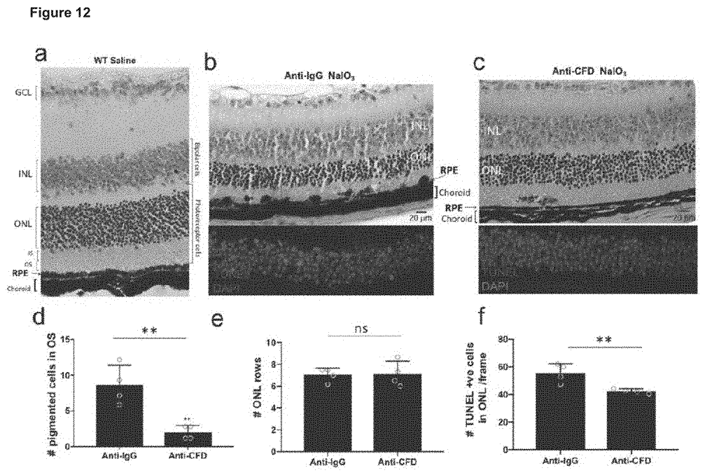

The NaIO₃ mouse model of oxidative stress mimics some features of human retinal disease albeit in an acute manner; notably complement deposition, RPE fragmentation and photoreceptor cell degeneration. In vitro, NaIO₃ dose dependently induced the expression of TLR2, CFB and C3 in RPE cells (FIG. 10), indicating the potential for activation of both TLR2 pathways and the AP in this model. Although there are potentially many DAMPs that drive TLR activation in response to oxidative damage, the appearance of CEP adducts was tested. FIG. 11 clearly demonstrates presence of CEP lipid oxidation product in the photoreceptor layer (3rd column) in NaIO₃ treated animals, when compared to saline and IgG-control sections (1^{st} and 2^{nd} columns). CEP appears strongly in the central retina, with weaker staining in peripheral retina, a phenomena also observed in a high glycemic diet-induced model of retinal degeneration. Surprisingly, evidence that AP activation has a role in promoting NaIO₃-induced disease has not been reported. To assess whether activation of AP is pathological in this model, sub-retinal injection of anti-CFD antibody, or anti-IgG control antibody was administered prior to IV NaIO₃. Use of anti-CFD neutralizing antibody was chosen to prevent CFD cleaving CFB resulting in inhibition of the amplification of the AP in the retina. H&E staining of a cross-section of a WT C57Bl/6 mouse eye administered saline by tail vein injection is provided for comparison (FIG. 12a). Marked fragmentation of the RPE was observed in mice injected IV with NaIO₃ and sub-retinal anti-IgG (FIG. 12b). In contrast, administration of anti-CFD blocking antibody rescued rupture of the RPE monolayer. A difference in the numbers of pigmented cells in the outer segments (OS) of the photoreceptor layers was also observed, with significantly fewer in the OS of eyes that had received anti-CFD treatment compared to anti-IgG controls (FIG. 12c). To assess the effect of anti-CFD treatment on photoreceptor cell death, the number of ONL rows for each treatment was counted. Despite rescue of the RPE, protection of rows of photoreceptors present in the ONL on neutralization of CFD (FIG. 12d) was not observed. However, it is possible that at a later time point this may be different, as TUNEL was significantly reduced in the anti-CFD blocking antibody group, compared with anti-IgG (FIG. 12e,f). The localization of TUNEL was notable as it appeared that in anti-CFD treated animals TUNEL was confined mainly to the inner layers of the ONL suggesting that perhaps the antibody did not achieve full perfusion into the photoreceptor layer.

### Example 5

### TLR2 Deficiency Protects Against NaIO₃ Induced RPE Fragmentation

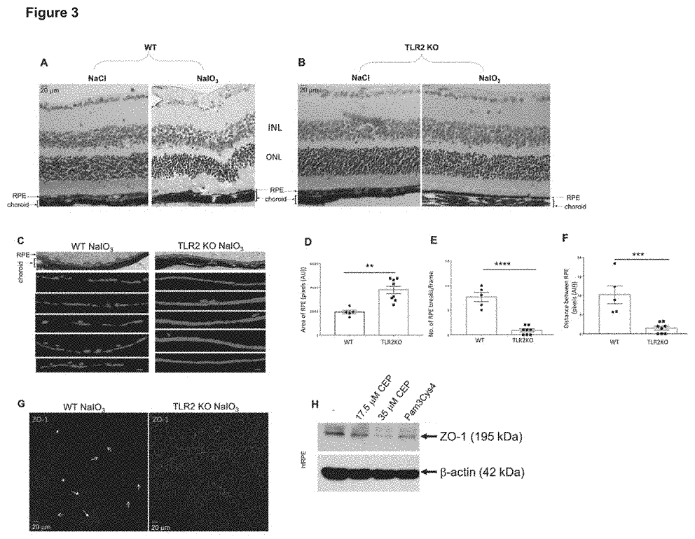

Having established that oxidative stress induced amplification of the AP is particularly damaging to the RPE in vivo, it was investigated whether TLR2 deficiency would modify this oxidative damage induced RPE fragmentation using TLR2 knockout (TLR2^{-/-}) mice. Marked degradation of the RPE was observed in the NaIO₃ group compared with vehicle NaCl group (FIG. 3a). NaIO₃ induced RPE fragmentation was similar in patterning to that observed in human tissue sections from AMD donor eyes (compare to FIG. 1h). In contrast, TLR2 deficiency substantially protected RPE degeneration and retinal structure following NaIO₃, although thinning of the RPE was still noted (FIG. 3b). The RPE constitutes the outer blood retinal barrier (oBRB), functioning to separate the neural retina from blood-borne plasma proteins, white blood cells and toxins. To quantify the protection observed in vivo, the area of the RPE shown in blue was selected and representative images from WT and TLR2^{-/-} mice 3 days post NaIO₃ (FIG. 3c, N>5) are presented and the area of the RPE/frame (FIG. 3d), number of breaks in the RPE/frame (FIG. 3e), and distance covered by RPE breaks/frame (FIG. 3f) assessed. All methods of analysis demonstrated significant protection to the RPE provided by TLR2 deficiency under oxidizing conditions. In addition to analysis of cross-sections of the RPE, RPE flatmounts from WT or TLR2^{-/-} mice were stained for tight junction protein ZO-1, 8 hours post NaIO₃ treatment. ZO-1 stained in the characteristic cobblestone pattern in TLR2 deficient mice (FIG. 3g, right hand panel). In direct contrast ZO-1 staining in WT mice was patchy, some areas demonstrated strong staining in a cobblestone pattern (FIG. 3g, left hand panel, closed arrows), however, there were also areas where no ZO-1 staining was apparent (FIG. 3g, left hand panel, asterisk's) and many areas where ZO-1 appeared to be at cell-cell junctions in an inconsistent, non-uniform pattern (FIG. 3g, left hand panel, open arrows). Indeed, when hfRPE cells were treated with TLR2 ligands CEP or Pam3Cys4, lysed and subjected to SDS-PAGE and immunoblotting, a marked decrease in ZO-1 expression was observed in all cases (FIG. 3h). The data indicate that absence of TLR2 signaling results in delayed oBRB breakdown in the NaIO₃ model of retinal degeneration. Interestingly, by 24 hours after NaIO₃ treatment, the cobblestone ZO-1 staining pattern is disrupted in both WT and TLR2^{-/-} mice (data not shown) and yet the RPE monolayer appears to remain more intact in the TLR2^{-/-} mice up to 3 days post NaIO₃, indicating the existence of other TLR2-dependent mechanisms at play in promoting RPE degeneration.

### Example 6

### TLR2 Deficiency Regulates C3 Deposition and Protects Against NaIO₃ Induced Photoreceptor Cell Death

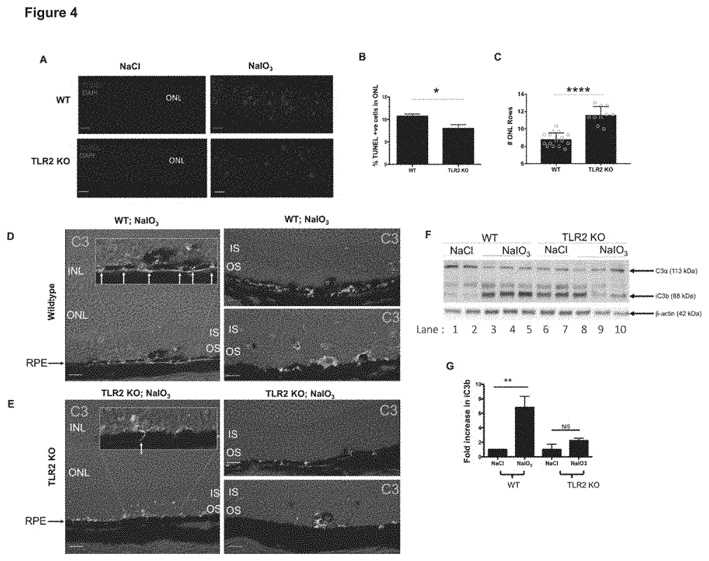

H&E staining of WT and TLR2^{-/-} eyes post NaIO₃ treatment indicated that the structure of the neural retina was better preserved in the TLR2^{-/-} mice (FIG. 3a, b). To assess the effect of TLR2 deficiency on photoreceptor cell death. TUNEL staining was utilized and which was significantly reduced in TLR2^{-/-} mice compared with WT (FIG. 4a, b). Furthermore, on counting rows of nuclei in the ONL it was found that TLR2 deficiency promoted survival of 2-3 rows of photoreceptor nuclei compared with WT mice (FIG. 4c). These data indicate that in addition to preserving the RPE monolayer, genetic loss of TLR2 protected photoreceptor neurons from oxidative stress-induced cell death. While it is possible that this preservation of photoreceptor cells is a result of loss of photoreceptor cell intrinsic TLR2-signaling, it is believed it more likely to be a secondary effect as a result of the improved preservation of the RPE and potentially a reduced C3 load in the outer retina. To investigate whether TLR2 deficiency effects C3 deposition in the NaIO₃ model in an analogous manner to the photo-oxidative stress model, total C3 in WT and TLR2^{-/-} mice (FIG. 4d, e) was stained for. C3 was observed in both WT and TLR2^{-/-} retinas post NaIO₃, however, C3 appeared to be differentially localized between WT and TLR2^{-/-} mice with more C3 at areas of the RPE where cell junctions were separating in the WT retina, and immediately basolateral to the RPE, compared with TLR2^{-/-} mice (FIG. 4d, e, left hand panels, insets, and right-hand top panels, arrows). C3 staining in the inner segments (IS) of WT retina with rarer occurrences in the TLR2^{-/-} retina was noted. Pigmented cells appeared more frequently in the WT photoreceptor OS compared with the TLR2^{-/-} retina and these cells were C3+(FIGS. 4d & 4e, right hand lower panel), however small focal points of C3 were observed in the OS of both WT and TLR2^{-/-} retinas. To examine activation of C3 in response to NaIO₃, RPE/choroid from WT or TLR2^{-/-} mice injected IV with NaIO₃ or NaCl were isolated and assayed for presence of the cleavage fragment iC3b. Upon activation, the thiol-ester bond in C3 is exposed, allowing covalent anchorage of C3b as well as its subsequent cleavage fragments to nearby molecules, iC3b is formed when CFI cleaves C3b in such a way that C3b cannot associate with CFB and instead functions as an opsonin. In lysate isolated from saline control WT mice C3α is observed, whereas iC3b is not present (FIG. 4f, N=2 lanes 1-2). In contrast, treatment with NaIO₃ resulted in a clear reduction in C3α and the appearance of iC3b indicative of C3 activation (FIG. 4f N=3, lanes 3-5, quantified in FIG. 4g). By comparison, there was no significant increase in iC3b formation in response to oxidative stress in the absence of TLR2 (FIG. 4f, g), instead the data indicate that TLR2 may have a homeostatic role in regulating C3 activity, as levels of baseline C3α are lower and levels of iC3b are higher in TLR2^{-/-} saline controls compared to saline controls in WT mice (FIG. 4f, compare lanes 1-2 to 6-7). Overall, the data indicate that DAMPs produced in response to oxidative stress engage TLR2 signaling to activate C3 cleavage and enhance opsonization of dying cells of the RPE.

### Example 7

### Oxidative Product CEP-HSA Induces MAC Formation on the RPE

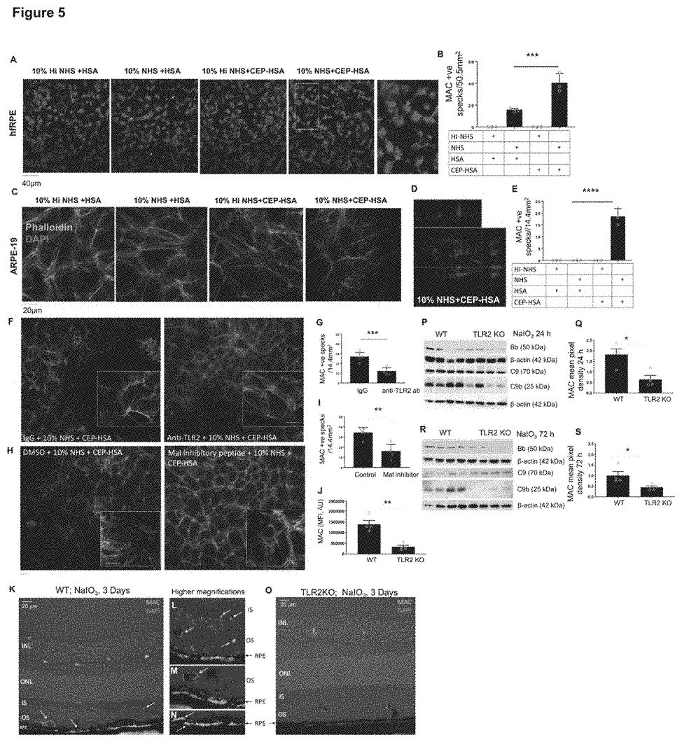

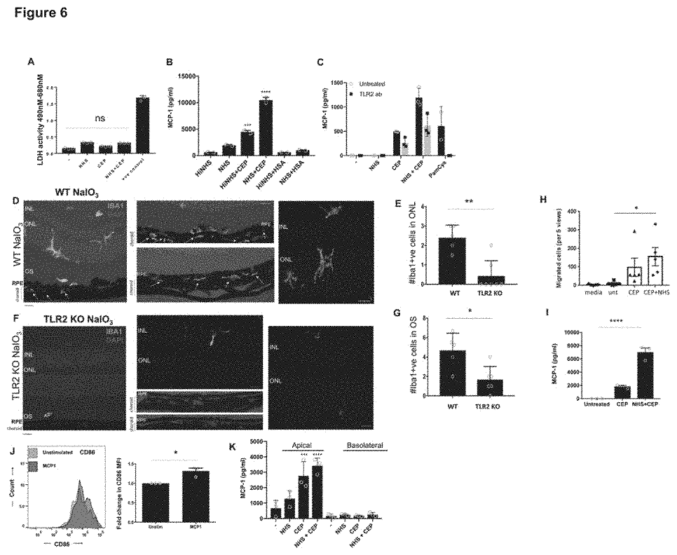

At this point, the data indicated that TLR2 plays a role in mediating C3 activation in response to oxidative stress resulting in the deposition of C3 and its opsonizing cleavage products in the outer retina. It was next investigated whether TLR2 can promote formation of the terminal complement complex MAC. CFB is the key rate limiting AP complement factor, as such, small increases in CFB expression, leads to formation of a C3 convertase (C3bBb) that amplifies the proteolytic cascade leading to C5 cleavage and ultimately formation of terminal complement MAC. During bacterial infection MAC usually leads to formation of a pore on the cell membrane, lysis and death of the bacteria. However, MAC is rarely lytic for nucleated cells and is reported to induce signaling pathways resulting in pro-inflammatory and pro-angiogenic gene expression on the RPE. Of particular interest is that known consequences of sub-lytic MAC formation are the release of chemokine monocyte chemoattractant protein CCL2/MCP-1 and vascular endothelial growth factor (VEGF), both cytokines are believed to have fundamental roles in promotion of dry and wet AMD respectively. It was suspected that the protective effect of TLR2 deficiency observed in the NaIO₃ model of retinal degeneration may be partially attributed to blockade of sub-lytic MAC formation and signaling in the RPE. To determine whether the presence of CEP with provision of complete complement was sufficient to drive the proteolytic complement cascade to completion in vitro, hfRPE were cultured on transwell membranes for >4 weeks prior to stimulation with either HSA or CEP-HSA in the presence of heat-inactivated (Hi) or normal human serum (NHS) for provision of complete complement. Culture of hfRPE cells in the presence of 10% NHS and HSA resulted in the appearance of visible MAC (FIG. 5a, white arrows, second panel), however culture of hfRPE cells in the presence of 10% NHS and CEP-HSA resulted in significantly more MAC being formed (FIG. 5a, fourth and fifth panels, FIG. 5b). hfRPE cells appeared to be less resistant to MAC formation in response to presence of 10% NHS than ARPE-19 cells, as CEP-HSA in the presence of serum proteins was the only combination that induced MAC formation on the membrane in the cell line (FIG. 5c-e). Quantification of MAC by ELISA also confirms CEP-HSA can significantly drive MAC formation in the presence of NHS (Supplementary FIG. 6). Of note, soluble MAC was also detected in cells cultured in NHS alone in both ARPE-19 cells and primary hfRPE cells despite a lack and near lack of membrane-associated MAC observed by confocal microscopy under these conditions. These data indicate that oxidative product CEP-HSA can promote the AP proteolytic cascade to completion with the embedding of MAC in the RPE membrane in vitro.

### Example 8

### Oxidative Stress-Induced TLR2 Activation Drives MAC Formation

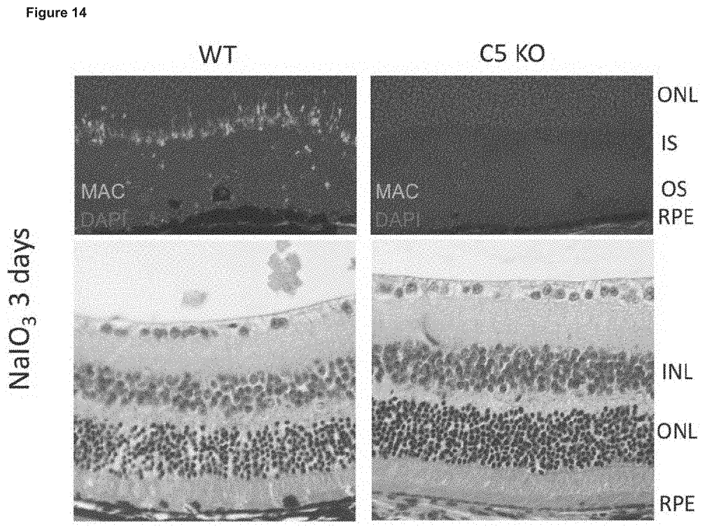

To confirm a role for TLR2 in the recognition of CEP-HSA and promotion of MAC in the RPE, the observation that ARPE-19 cells formed membrane-embedded MAC only in the presence of 10% NHS and CEP-HSA, with no visible membrane-embedded MAC formed in the presence of 10% NHS or 10% NHS+HSA alone (FIG. 5c), allowed these cells to be used as a tool to examine CEP-HSA inducible MAC specifically. RPE cells treated with NHS and CEP-HSA in the presence of a monoclonal neutralizing anti-TLR2 antibody or an isotype control (IgG) were assayed for MAC formation by confocal microscopy (FIG. 5f, g). Neutralizing TLR2 attenuated the number of MAC formed in response to CEP-HSA by approximately 50% (FIG. 5f, g). To further confirm the role of TLR2 in bridging oxidative stress to MAC formation, it was investigated whether inhibiting TIR adaptor Mal would affect CEP-HSA induced MAC formation. RPE cells were cultured with NHS and CEP-HSA in the presence of a Mal inhibitor peptide or peptide control and MAC formation was assayed by confocal microscopy (FIG. 5h, i). Inhibiting Mal significantly attenuated the number of MAC specks formed in response to CEP-HSA, in an analogous manner to TLR2 neutralization (FIG. 5h, i). Together this data confirms that TLR2 acts as a bridge between AMD-associated lipid oxidation product CEP and the induction of AP-driven MAC formation. However, given the inhibition of MAC formation after TLR2/Mal blockade is not complete, TLR2/Mal-independent signaling pathways are also implicated in contributing to CEP-induced MAC formation. MAC is known to be rapidly endocytosed from the RPE membrane in vitro and is rarely observed on the RPE in human tissue samples. In fact, outside of MAC induced pathology of the choriocapillaris, a clear link between MAC deposition in the retina and retinal degeneration is not firmly delineated. To determine whether MAC formation may be involved in oxidative stress induced retinal pathology, MAC/C5b-9 was examined by IHC in C5 deficient mice compared with WT mice 3 days after IV injection with NaIO₃ (FIG. 14). MAC/C5b-C9 was observed in the IS and OS of the photoreceptors in WT tissue sections (FIG. 14 top left panel), surrounding pigmented cells in the OS layer, and immediately adjacent to the RPE. MAC/C5b-C9 was not observed in the retina of C5 deficient mice (FIG. 14, top right panel) and H&E staining demonstrated that in addition to lacking MAC deposition, C5 deficiency also protected from oxidative stress induced photoreceptor degeneration (FIG. 14). Activation of C5 generates anaphylatoxin C5a and MAC-forming C5b products. While the possibility exists that the protection observed could be due to loss of either C5a or C5b alone, as both products are generated simultaneously in parallel it is likely that loss of both contribute to the protection observed in response to oxidative stress, pointing to a role for MAC in this model of retinal degeneration. Next, the localization of MAC deposition 3 days post NaIO₃ treatment in the WT and TLR2^{-/-} mice (FIG. 5j-k-o) was examined MAC was observed in the IS and OS of the photoreceptors in WT tissue sections (FIG. 5k), surrounding pigmented cells in the OS layer, and immediately adjacent to the RPE both apical and basolateral the RPE membrane (FIG. 5l-n high magnification). In contrast, MAC was not observed in the retina of TLR2^{-/-} mice (FIG. 5o). (Note: this is a mouse IgG antibody, therefore the green fluorescence in the INL is indicative of the inner blood retinal vasculature). As CFB active fragment Bb is required to form the C5 convertase the appearance of active Bb and C9/C9b MAC products in retinal lysate from WT and TLR2^{-/-} mice 1 day (FIG. 5p, q) or 3 days (FIG. 5r, s) post NaIO₃ was investigated. Across both timepoints, Bb appeared more abundant in WT mice compared with TLR2^{-/-} mice post NaIO₃ treatment (FIG. 5p, r, top panels). Zymogen C9 (70 kD) is proteolytically cleaved at a specific site in order in induce C9 polymerization. The 25 kD product, which is the carboxyl-terminal fragment of C9 capable of disturbing membrane potential, was found to a strikingly greater extent in the WT mice compared with the TLR2^{-/-} mice at both 1 day (FIG. 5p, q) and 3 days (FIG. 5r, s) post NaIO₃ treatment. These data imply that TLR2 signaling drives the AP cascade to completion with formation of MAC in response to oxidative stress in vivo.

### Example 9

### Oxidative Product CEP-HSA Induces Sub-Lytic MAC Formation on the RPE and Secretion of Chemokine MCP-1

To interrogate whether CEP/NHS induced MAC formation on the RPE was lytic or sub-lytic, a lactate dehydrogenase (LDH) assay was used as an indicator of cell death and an MCP-1/CCL2 ELISA as an indicator of sub-lytic MAC signaling. The RPE supernatant was harvested from the MAC-assay (FIG. 5c) and no apparent cell death of RPE cells was observed under conditions where MAC was forming (ie. CEP-HSA+NHS) (FIG. 6a); yet in the same samples a significant increase in the secretion of MCP-1/CCL2 from cells with sub-lytic MAC formation (CEP-HSA+NHS) was observed over cells treated with NHS or CEP-HSA alone (FIG. 6b). Neutralization of TLR2 signaling using anti-TLR2 under the same conditions demonstrated a halving of CEP- and CEP/NHS-induced MCP-1/CCL2, in contrast Pam3Cys4-induced MCP-1/CCL2 was abolished in the absence of TLR2 signaling (FIG. 6c). The lack of complete penetrance of neutralizing TLR2 on CEP-induced MCP-1/CCL2, mirrors the incomplete inhibition of MAC formation observed with TLR2 neutralization (FIG. 5f-i) and implies the existence of both TLR2-dependent and -independent components to CEP signaling. However, collectively these data imply that TLR2 can act as a sensor for oxidative stressor CEP, and that CEP-TLR2 signaling synergizes with complement resulting in sub-lytic MAC formation that functions to generate a chemokine signal from the RPE rather than cause cell lysis.

### Example 10

### TLR2 Deficiency Delays NaIO₃ Induced Iba1+ Macrophage/Microglial Infiltration to the Outer Retina

TLR2 deficiency has been reported to reduce macrophage infiltration in the CNS in response to spinal nerve injury. The retina is an extension of the CNS and MCP-1/CCL2 is a potent chemoattractant and the major chemokine responsible for macrophage and microglial infiltration in the retina. The implication is that TLR2 deficiency may result in reduced macrophage and microglial infiltration to the retina in response to oxidative stress. A recent report has demonstrated that photoreceptor cell death in the NaIO₃ model is correlative with activated macrophage accumulation in the outer retina following RPE degeneration. The extent to which absence of TLR2 might influence macrophage/microglial cell migration into the outer retina was next assessed in response to oxidative stress. IHC for Iba1 72 hours post NaIO₃ was assessed. Iba1 stains both macrophages and microglia and by 72 hours large Iba1+ cells were found both in the ONL (FIG. 6d, e) and in among the OS of WT mice immediately apical to RPE (FIG. 6d, g). Large Iba1+ cells were also observed appearing subjacent to RPE (FIG. 6d, middle panels). There were significantly fewer Iba1+ cells in the ONL or OS of TLR2^{-/-} mice (FIG. 6d-g lower panels). These data indicate that oxidative stress induced TLR2 signaling drives a chemokine gradient in vivo that has the potential to regulate both the migration of microglia from the inner to the outer retina and the migration and infiltration of myeloid cells from the choroid to the outer neural retina. In vitro, it was investigated whether conditioned media from RPE cells treated under CEP+NHS sub-lytic MAC conditions would affect monocyte migration across a transwell as a proxy for understanding whether blockade of sub-lytic MAC signaling in RPE cells in vivo might reduce macrophage/microglia cell infiltration analogous to what was observed in the NaIO₃ treated TLR2^{-/-} mice. Increased cell migration across a transwell membrane towards the chamber was observed with media transferred from RPE cells when compared with controls (FIG. 6h), MCP-1 levels in the transferred media are shown for context (FIG. 6i). Next, the effect of MCP-1 on CD86 as a proxy for monocyte activation was assessed. MCP-1 treatment of peripheral blood mononuclear cells (PBMC's) increased CD86 membrane expression by ˜1.4 fold (FIG. 6j), indicating not only that sub-lytic MAC formation on RPE cells has potential to provide a chemokine gradient for monocytes but also that the monocytes may be further activated by the environment. The IHC data indicated that in addition to the decreased infiltration of Iba1+ve cells from the inner retina towards the outer retina, that there is a deficit of macrophages infiltrating from the choroidal vasculature towards the outer retina also in TLR2^{-/-} mice (FIG. 6d, f middle panels). RPE cells are polarized cells so the extent to which RPE cells secreted MCP-1/CCL2 apically versus basolaterally was investigated. Interestingly, it was found that RPE cells cultured on transwells for >5 weeks secrete MCP-1/CCL2 in a strikingly polarized manner when treated with CEP or CEP+NHS, with no significant secretion into the basolateral compartment compared with strong cytokine release into the apical compartment (FIG. 6k). These data imply that while CEP and sublytic MAC signaling likely play a role in the infiltration of Iba1+ cells from the inner retina, there is likely an additional chemotactic signal that attracts macrophages from the choroid. The proteolytic complement cascade ending in MAC formation concomitantly generates soluble anaphylatoxins C3a and C5a. And despite being rapidly turned over, C5a in particular is a potent chemotactic agent for monocytes, macrophages and microglia, it is possible that C5a is also involved in drawing Iba1+ cells into the retina in response to oxidative stress although this remains to be tested.

### Example 11

### Anti-TLR2 Therapy Protects Against NaIO₃ Induced RPE Degeneration and Photoreceptor Cell Death

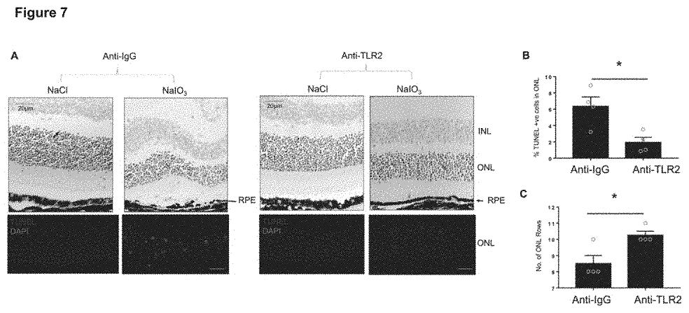

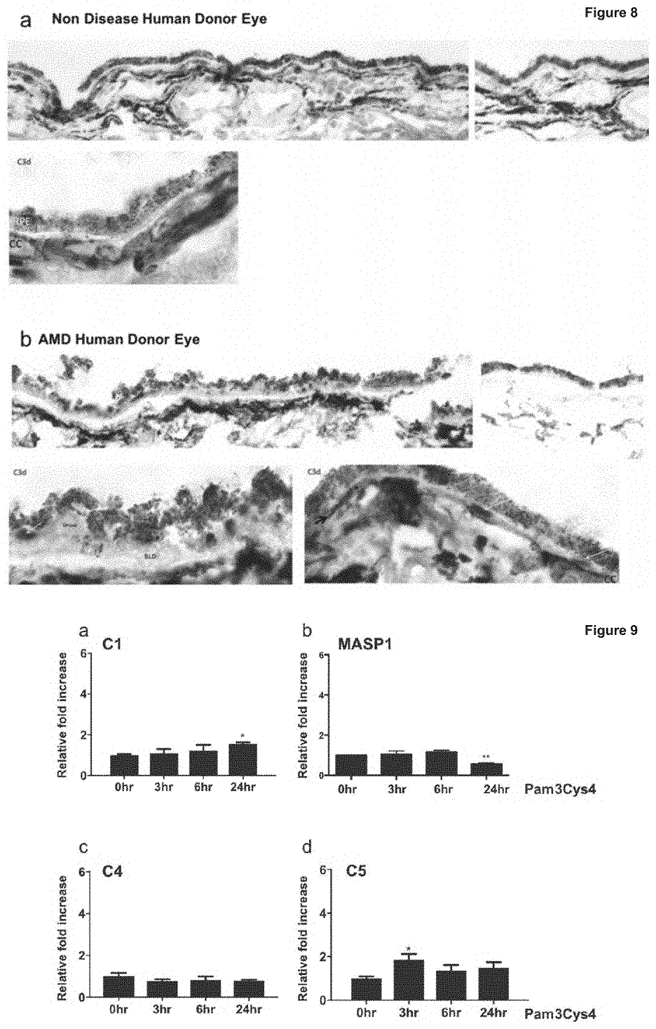

Anti-TLR2 neutralizing antibodies had preserved photoreceptor degeneration in the focal photo-oxidative stress induced model of retinal degeneration. It was next investigated whether pharmacological blockade of TLR2 signaling, through use of the same anti-TLR2 neutralizing antibody would rescue RPE fragmentation and photoreceptor degeneration in the NaIO₃ model and could therefore broadly present TLR2 as a therapeutic target for oxidative stress induced retinal degeneration. WT mice were injected IV with NaIO₃ or with vehicle NaCl and with sub-retinal anti-IgG or anti-TLR2 antibodies and retinal histology was assessed 72 hours after NaIO₃. As expected, marked fragmentation of the RPE was observed in mice injected IV with NaIO₃ and sub-retinal anti-IgG compared with mice injected IV with vehicle NaCl and sub-retinal anti-IgG (FIG. 7a top panels). In contrast, sub-retinal administration of anti-TLR2 blocking antibody into both eyes at time of tail vein NaIO₃ administration rescued RPE degeneration and retinal structure (FIG. 7a, top panels). To assess the effect of anti-TLR2 treatment on photoreceptor cell death we utilized TUNEL staining. TUNEL staining was significantly reduced in mice that received anti-TLR2 blocking antibody, in comparison with those that received anti-IgG (FIG. 7a bottom panels, 7b). Specifically, analogous to the TLR2^{-/-} mice, it was found that therapeutic neutralization of TLR2 also protected 2-3 rows of photoreceptors from oxidative stress-induced cell death (FIG. 7c) and preserved the RPE monolayer post administration of NaIO₃. Collectively these data demonstrate that administration of a neutralizing anti-TLR2 antibody leads to a protection of the RPE, and a reduction in photoreceptor cell death under oxidative conditions.

### Discussion

With progressive age, increased oxidative damage occurs in many tissues, including the retina, and is thought to contribute to the progression of multiple forms of retinal degeneration most notably AMD. This is highlighted in a variety of experimental models where increased oxidative stress leads to a dry AMD-like pathology, including immunization with CEP, knockdown of SOD2 and NaIO₃ injection. In addition to excessive oxidative stress an accumulation of complement factors in the retina and choroid is a pathological hallmark of AMD. It has been suggested that products of photo-oxidation of bis-retinoid lipofuscin pigments could serve to activate complement. However, the underlying mechanisms that trigger complement fixation in response to oxidative stress remain unknown. CEP has been shown to act as a ligand for TLR2 promoting angiogenesis in response to oxidative stress and indeed blocking TLR2 signaling in two mouse models of choroidal neovascularization (CNV) was recently shown to be efficacious in reducing CNV lesion size. This indicates that inhibitors of TLR2 have potential therapeutic utility for wet AMD.

It was chosen to study the effect of neutralizing TLR2 in two different experimental models of retinal degeneration. While both models utilized are oxidative stress induced models of retinal degeneration known to deposit complement and result in loss of photoreceptor cells, the major cell types effected in each model differ. In the photo-oxidative stress model, C3 is microglia/macrophage derived, deposited in the outer segments, and has been shown to contribute causally to photoreceptor loss. However, despite reports of C3 accumulation, no causative role for the AP had been implicated in retinal degeneration in the NaIO₃ model. In order to determine whether AP activation was a driver of the pathology observed in the NaIO₃ model or simply a bystander effect, an immunoprecipitating blocking antibody for Complement factor D (CFD) was used. CFD is a serine protease that cleaves CFB once bound to C3b, resulting in the assembly of the AP C3 convertase. An interesting observation relating to the use of anti-CFD in the NaIO₃ model was the resulting protection of the RPE, with no significant loss of photoreceptor numbers. CFD binds to C3 only after it has bound CFB, at which point it cleaves CFB and enables amplification of the AP. For this reason, introduction of anti-CFD will block the amplification step of the AP, inhibiting MAC formation, but its inhibition of C3 cleavage into opsonizing fragments is less effective. With this in mind the anti-CFD data indicates that photoreceptors are sensitive to C3 deposition/opsonisation whereas the RPE may be more sensitive to the effects of amplifying the AP. Indeed, others have shown that complement regulators Cd55/Cd59 are reduced specifically in the photoreceptors in a model of retinal detachment, making photoreceptors especially sensitive to opsonization and complement-mediated death. By contrast, the fact that TLR2 deficiency protected both photoreceptor numbers and the RPE implies that, in response to oxidative stress, TLR2 signalling promotes both C3 opsonisation and the amplification of the AP. Indeed, CFB is exclusive to the AP and is the key rate limiting protein in AP activation. Simply increasing CFB expression can lead to the formation of the C3 convertase, activating the AP by cleaving C3. TLR2 activation consistently induced gene expression of both CFB and C3 to significant levels in all cell types tested, implying that TLR2 activation can universally activate the AP in vitro. Likewise, in vivo, we observed C3 opsonin fragment deposition in response to oxidative stress was lessened in the absence of TLR2. It is worth noting that, CFH functions to inhibit the amplification of the AP by competing with CFB for binding with C3. In this way variants in CFH that heighten risk for dry AMD and progression to GA are less efficient at preventing the amplification of the AP, again indicating a sensitivity of the RPE to the effects of the amplification of the AP. Amplification of the AP leads to terminal complement activation; whereupon its individual components C5b, C6, C7, C8, and C9 combine to form a lytic pore (C5b-9/MAC) on the surface of target cell membranes, capable of inducing cell lysis and inflammatory processes, as well as activating various cell signaling pathways. In the human retina, the MAC complex is identified in Bruch's membrane in eyes as young as 5 years of age. The presence of MAC increases with normal ageing, but it accumulates at higher levels in individuals with risk-associated AMD genotypes and has been identified in AMD patients within drusen in Bruch's membrane surrounding the choriocapillaris, and on RPE overlying drusen in vivo. The fact that the RPE is more intact in the absence of TLR2, despite being subjected to oxidative stress, implies that amplification of the AP has been inhibited due to the loss of TLR2 signalling. Indeed, the lack of active MAC formation in response to oxidative stress in the retina, in the absence of TLR2 was marked when compared to WT mice. Previous reports demonstrate that inhibition of TLR2 reduces C3 deposition in ischemia-reperfusion injury, these data demonstrate that TLR2 can also directly trigger the proteolytic complement cascade to completion with formation of the terminal complement complex, MAC.

MAC activation on choroidal endothelial cells induces lysis but studies describe how RPE cells are resistant to MAC mediated lysis and efficiently remove MAC before lysis can take place; instead, sub-lytic MAC induces inflammatory signaling pathway activation. In support of these reports, RPE cell death under TLR2 induced MAC-forming culture conditions were not observed in vitro, indicating that TLR2-induced MAC formation on RPE cells is sub-lytic. Sub-lytic MAC is characterized by secretion of MCP-1/CCL2, a key monocyte chemoattractant that also signals for monocyte differentiation into macrophages. In line with this characteristic, a synergistic effect on MCP-1/CCL2 secretion was observed under culture conditions where TLR2 induced MAC is formed, above the induction observed in response to CEP alone, suggesting that MAC formed on RPE cells in response to TLR2 activation is sub-lytic and has the potential to create an environment that attracts phagocytes to the outer retina. Interestingly, MCP-1 secretion from the RPE was highly polarized favoring a role for resident microglia activation in the neural retina. From a mechanistic stand point, support for MCP-1/CCL2 as a major factor in recruiting phagocytes in retinal degeneration comes from reports that genetic deletion of MCP-1/CCL2 prevents inflammatory monocyte recruitment, accumulation and photoreceptor degeneration in vivo in mouse models. The decreased Iba1+ staining and photoreceptor degeneration we observed in TLR2 deficient mice after treatment with NaIO₃ supports the existence of a TLR2-driven chemokine gradient, attracting these cells to the outer retina and contributing to photoreceptor cell death, which may be a consequence of sub-lytic MAC signaling, although this remains to be definitively tested.

These data indicate that TLR2 mediates complement deposition in response to oxidative stress that is pathological in nature, and that blocking TLR2 signaling preserves both photoreceptor and RPE integrity in vivo under conditions of acute oxidative stress. However, the respective contributions of the different cells in the retina that can respond to TLR2 and their individual contributions to oxidative stress-induced TLR2 promotion of retinal degeneration requires further examination. It cannot yet be distinguished between relative contributions to pathology made by TLR2-activated RPE and TLR2-activated mononuclear phagocytes. The observation that neutralizing TLR2 in the photo-oxidative damage model of retinal degeneration, significantly reduced complement deposition and preserved photoreceptor cell layers, indicates that in addition to RPE-originating signals, blocking TLR2 signaling in the macrophage/microglia cells is also likely to contribute a significant aspect to the prevention of TLR2-mediated retinal degeneration. Furthermore, given these data, and supporting literature, that MAC formation on the RPE is sub-lytic, it remains to be understood how oxidative stress results in RPE fragmentation in vivo and following this why blocking TLR2 signaling in response to oxidative stress delays the RPE from this degeneration. Others have demonstrated that merely overexpressing C3 alone in vivo with C3-expressing adenovirus exhibited similarly significantly increased RPE death, in addition to loss of photoreceptor outer segments, and reactive gliosis. So it appears that, in vivo, unregulated complement activation results in an environment that promotes RPE death, be it as a result of experimental AAV-inducible C3 overexpression, or in our case oxidative damage-induced TLR2-mediated C3/MAC activation. Importantly, during the course of this study, it was also discovered TLR2 effects on the RPE that are independent of its role in inducing complement but undoubtedly contribute to RPE degeneration. Specifically, oxidative stress induced TLR2 signalling can also reduce tight junction expression, likely contributing to weakening the RPE and consequently the outer blood retinal barrier.

In conclusion, we show that TLR2 deficiency reduces complement activation, delays oxidative damage induced RPE fragmentation, delays migration of microglia/macrophages to the RPE and outer neural retina, and delays photoreceptor degeneration. These data contribute towards understanding the mechanisms underlying oxidative stress induced retinal degeneration and pinpoints TLR2 as a PRR bridging the detection of oxidative damage to activation of the complement response providing new targets for the prevention of oxidative stress induced pathology.

TLR2 heterodimerises with either TLR1 or TLR6 and recognizes diacyl and triacylated lipopeptides. TLR2 and TLR4 protect against infection in the anterior region of the eye. However, investigations into roles for TLRs in outer retinal disease are sparse, and mainly confined to genetic investigations, including several contradicting reports of associations between various SNPs in TLRs and risk of AMD. TLR signaling and the complement system have been linked in intestinal ischemia-reperfusion injury, where C3 deposition was markedly decreased in mice deficient in TLR4, and in a renal transplant ischemia-reperfusion injury model, where inhibition of TLR2 led to a decrease in C3 deposition. Furthermore, activation of TLR4 and TLR2 increases C1-13 expression in macrophages. TLR function has not been explored in outer retinal degenerative disease and RPE pathology. Here, it was sought to explore whether TLR2 might act as a bridge between effects of oxidative stress and complement activation in the retina. In doing so, it was discovered that TLR2 inhibition provides striking protection to the retina in response to oxidative stress. It is shown that oxidative stress activates TLR2 to trigger the proteolytic alternative pathway (AP) to completion with generation of the terminal complement complex, that forms sub-lytic MAC on the RPE and induces the pro-inflammatory chemokine MCP-1/CCL2. It is demonstrated that inhibition of TLR2 reduces complement activation, C3 opsonization and MAC deposition, ameliorates RPE fragmentation, prevents Iba1+ve macrophage/microglial cell infiltration to the outer retina, and preserves photoreceptor cells in response to acute oxidative stress. These data suggest that TLR2 signaling promotes an environment that drives a retinal degenerative phenotype, and presents TLR2 as a possible link between oxidative damage and excessive complement activation in retinal degenerative disease.

Retinal degeneration is a form of neurodegenerative disease and is the leading cause of vision loss globally. The Toll-Like Receptors (TLRs) are primary components of the innate immune system involved in signal transduction. The present invention shows that TLR2 induces complement factors C3 and CFB, the common and rate limiting factors of the Alternative Pathway in both retinal pigment epithelial (RPE) cells and mononuclear phagocytes. Neutralisation of TLR2 reduces opsonising fragments of C3 in the outer retina and protects photoreceptor neurons from oxidative stress-induced degeneration. TLR2 deficiency also preserves tight junction expression and promotes RPE resistance to fragmentation. Finally, oxidative stress-induced formation of the terminal complement membrane attack complex and Iba1+ cell infiltration are strikingly inhibited in the TLR2 deficient retina. These data directly implicate TLR2 as a mediator of retinal degeneration in response to oxidative stress and present TLR2 as a bridge between oxidative damage and complement-mediated retinal pathology.

1. A method of treating age-related macular degeneration in a subject in need thereof, the method comprising the step of decreasing the expression or activation of a toll-like receptor in the subject.

2. The method of claim 1, wherein the toll-like receptor is selected from the group consisting of TLR1, TLR2, TLR3, TLR4, TLR5, TLR6, TLR7, TLR8, TLR9, TLR10, TLR11, TLR12, and TLR13.

3. The method of claim 1, wherein toll-like receptor is TLR2.

4. The method of claim 1, wherein the age-related macular degeneration is selected from the group consisting of dry age-related macular degeneration, non-exudative age-related macular degeneration, and non-neovascular age-related macular degeneration.

5. The method of claim 1, wherein the method comprises the step of administering a pharmaceutically effective amount of an antagonist of a toll-like receptor to the subject to decrease the expression or activation of the toll-like receptor and so treat age-related macular degeneration in the subject.

6. The method of claim 1, wherein the method comprises the step of administering an agent capable of decreasing expression of the toll-like receptor.

7. The method of claim 6, wherein the agent is selected from the group consisting of antisense oligonucleotides, ribozymes, small interfering RNAs (siRNA), microRNA (miRNA), small/small hairpin RNA (shRNA), and nucleic acid aptamers.

8. The method of claim 6, wherein the agent is a deoxyribonucleic acid aptamer.

9. The method of claim 6, wherein the agent is selected from the group consisting of a retrovirus-, adenovirus-, herpes simplex-, vaccinia-, and adeno-associated virus-delivered vector.

10. The method of claim 6, wherein the agent is delivered by the group consisting of injection of naked DNA, electroporation, the gene gun, sonoporation, magnetofection, the use of oligonucleotides, lipoplexes, dendrimers, and inorganic nanoparticles.

11. The method of claim 1, wherein the method comprises the step of administering an agent capable of decreasing the activation of the toll-like receptor.

12. The method of claim 11, wherein the agent is a toll-like receptor antagonist selected from the group consisting of a competitive toll-like receptor antagonist, a non-competitive toll-like receptor antagonist, an uncompetitive toll-like receptor antagonist, a silent toll-like receptor antagonist, and an inverse toll-like receptor agonist.

13. The method of claim 11, wherein the agent is a toll-like receptor antagonist selected from the group consisting of a reversible toll-like receptor antagonist, and an irreversible toll-like receptor antagonist.

14. The method of claim 11, wherein the agent is a toll-like receptor antagonist selected from the group consisting of a selective toll-like receptor antagonist, and a non-selective toll-like receptor antagonist.

15. The method of claim 11, wherein the agent is a toll-like receptor antagonist selected from the group consisting of a chemical compound toll-like receptor antagonist, a small molecule toll-like receptor antagonist, an immunoglobulin toll-like receptor antagonist, and a lipid-A analogue toll-like receptor antagonist.

16. The method of claim 5, wherein the method comprises administering the toll-like receptor antagonist to the retinal pigment epithelium.

17. The method of claim 16, wherein the method comprises administering the toll-like receptor antagonist to cells selected from the group consisting of retinal microglia cells, muller glia cells and mononuclear phagocytes.

18. The method of claim 1, wherein the method comprises the further step of decreasing the expression or activation of Myeloid differentiation primary response 88 (MYD88) in the subject.

19. The method of claim 1, wherein the method comprises the further step of decreasing the expression or activation of MyD88-adapter-like (Mal) in the subject.
